(19) United States
(12) Patent Application Publication
(10) Pub. No.: US 2019/0047939 A1
(43) Pub. Date: Feb. 14, 2019

(54) PROCESS AND INTERMEDIATES FOR THE PRODUCTION OF 17(20)-ENE B-SECO STEROIDS

(71) Applicant: Patheon Austria GmbH & Co KG, Linz (AT)

(72) Inventors: George Petros YIANNIKOUROS, Florence, SC (US); Panos KALARITIS, Florence, SC (US); Denis Viktorovich AREFYEV, Florence, SC (US)

(73) Assignee: Patheon Austria GmbH & Co KG, Linz (AU)

(21) Appl. No.: 16/080,358

(22) PCT Filed: Mar. 1, 2017
(86) PCT No.: PCT/EP2017/054838

§ 371 Date: Aug. 28, 2018

Related U.S. Application Data
(60) Provisional application No. 62/302,410, filed on Mar. 2, 2016.

(51) Int. Cl. C07C 213/08; C07C 45/29; C07C 41/01; C07C 249/02; C07J 21/00; C07C 62/26; C07C 251/06; C07C 217/52

(52) U.S. Cl. CPC C07C 213/08; C07C 45/29; C07C 41/01; C07C 249/02; C07C 2602/24; C07C 62/26; C07C 251/06; C07C 217/52; C07C 2601/14; C07J 21/008

(57) ABSTRACT

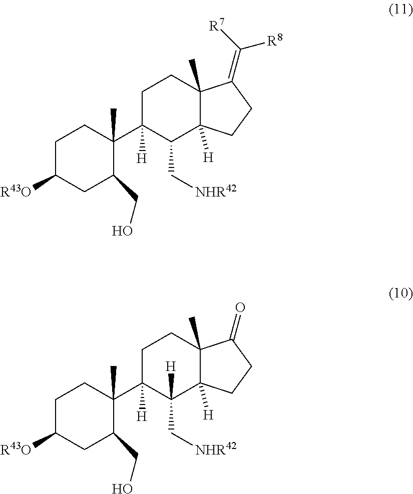

The invention pertains to a process for producing a compound of formula (11) wherein R⁷ and R⁸ are each independently selected from H, halogen, alkyl, aryl, or alkylaryl, R⁴² is H or a protective group, R⁴³ is H or R³, wherein R³ is a protective group, by contacting a compound of formula (10) with an olefmation reagent, wherein compound of formula (10) comprises a counter acid X¹ when R⁴²═H and R⁴³═H.

The present invention relates to a process for the production of secosteroids, in particular octahydro-1H-indene derivatives and further pertains to novel intermediates prepared in the process of the invention and to the use of the compounds produced with the process of the invention in a process for the preparation of an active pharmaceutical ingredient, for example an active pharmaceutical ingredient useful for the treatment of inflammatory diseases, bladder pain syndrome/interstitial cystitis and related disorders.

### BACKGROUND OF THE INVENTION

Secosteroids, in particular octahydro-1H-indene derivatives, more in particular 5-cyclohexyloctahydro-1H-indene derivatives are well known as anti-inflammatory agents (WO 2004092100) and for the treatment of disorders or conditions that would benefit from SHIP1 (SH2 domain-containing inositol phosphatase) modulation, e.g. immune and hemopoietic disorders (WO 2014143561). A target compound which is of particular interest in this respect is (1S,3S,4R)-4-((3aS,4R,5S,7aS)-4-(aminomethyl) -7a-methyl-1-methyleneoctahydro-1H-inden-5-yl)-3-(hydroxymethyl)-4-methylcyclohexan-1-ol acetate.

Known synthetic methods for the preparation of 5-cyclohexyloctahydro-1H-indene derivatives which can act as SHIP1 modulators can be performed starting from a number of steroid percursors including 4-androsten-3,17-dione and others with C3 oxygen functionalities and Δ⁵ carbon-carbon double bonds such as dehydroisoandrosterone as described in U.S. Pat. Nos. 6,046,185, 7,601,874 and WO 2014/143561.

The disadvantage of the known synthetic methods is the process complexity, i.e. considerably more than 10 steps are needed to obtain the desired secosteroids when starting from the aforementioned steroid precursors to obtain the desired secosteroids, and several chromatographic purification steps are required. The overall yields of the known processes are generally low.

The object of the present invention is to provide an alternative process.

### SUMMARY OF THE INVENTION

The object of the invention is achieved by a process for producing a compound of formula (11)

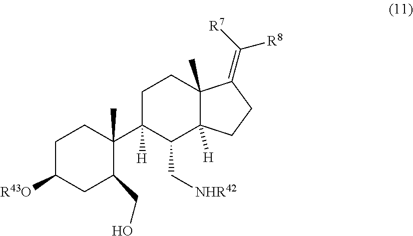

wherein R⁷ and R⁸ are each independently selected from H, halogen, alkyl, aryl, or arylalkyl, R⁴² is H or a protective group, R⁴³ is H or R³, wherein R³ is a protective group, by contacting a compound of formula (10)

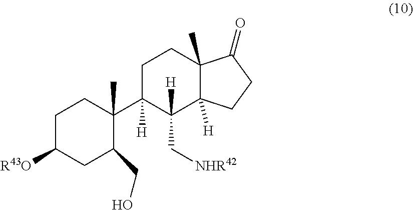

with an olefination reagent, wherein compound of formula (10) comprises a counter acid X¹ when R⁴²═H and R⁴³═H.
The process of the invention enables an alternative process for the production of the compound of formula (11). The alternative process generally allows for less process steps compared to conventional processes. The process is generally less time-consuming, less complex, more cost efficient and increases flexibility. A further advantage of this process is that the compound of formula (11) can be isolated as a solid.
One embodiment of the process of the present invention may be exemplified by the following scheme:

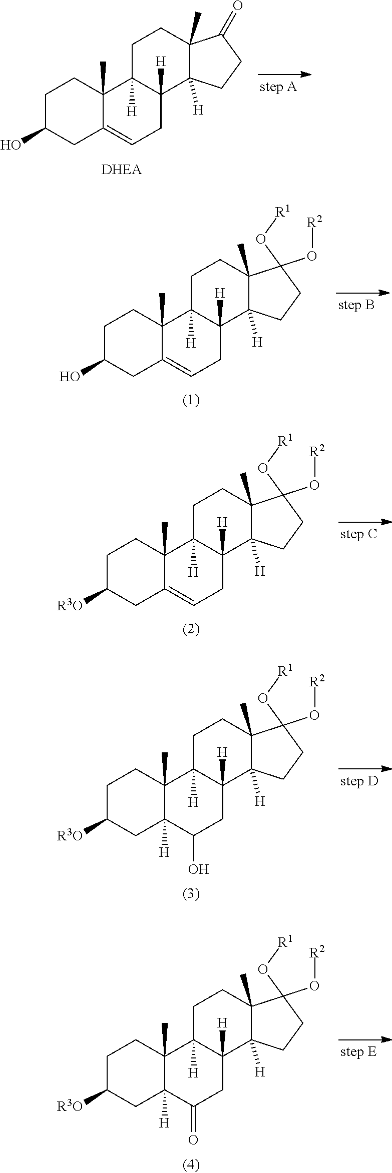

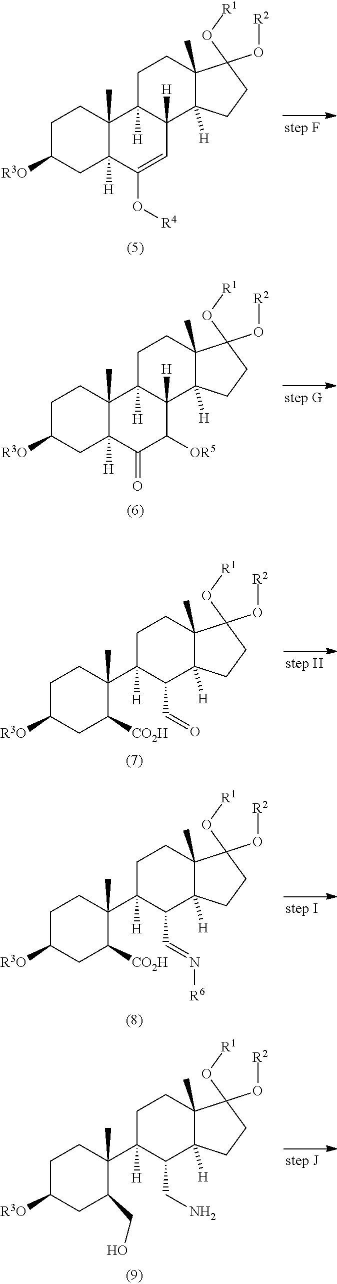

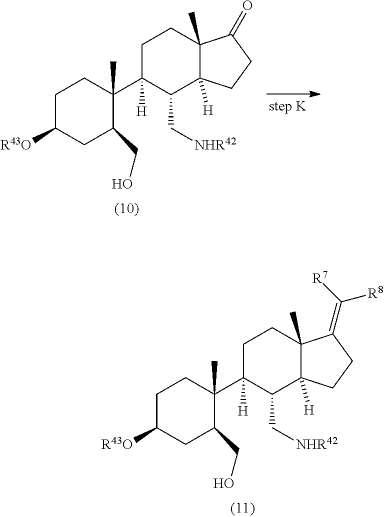

Some of the conversion steps, in particular steps A and B, C and D, E to G, and H to J, can be performed without intermediate isolation. This generally renders the process of the invention to be less time-consuming, less complex and more cost efficient. U.S. Pat. No. 6,046,185 and WO 2004/092100 when combined disclose a synthetic pathway to produce a compound of formula (12) from dehydroepiandrosteron (DHEA). This pathway generally requires considerably more steps than the process of the invention. Typically overall yields of the present invention are similar or higher than the yield from conventional synthetic pathways. The process of the invention allows for considerably less purification steps using chromatography compared to conventional processes. Such purification steps are laborious and generally render the process not to be time and/or cost efficient. The process of the invention enables a process absent of such chromatographic purification steps. Chromatographic purifications usually have disadvantages in that a significant amount of eluents is necessary for their operation and that they may limit the throughput of a process. Moreover the reprocessing of the mobile phase may be energy-intensive and the stationary phase may cause significant amounts of waste. In general the “Material input per unit of service” (MIPS) value of chromatographic purification to reach a desired product quality is significant. With the process of the invention the compound of formula (12) can easily be obtained at a purity level which is generally higher compared to conventional processes. A further advantage of the process of the present invention is that all intermediates may be readily isolated as solids, which generally allows for a good storage stability as well as easy transport enabling the performance of the process at two or more production facilities at different locations.

Compound of formula (11) may be further contacted with a second acid which forms a non-toxic salt to obtain a compound of formula (12)

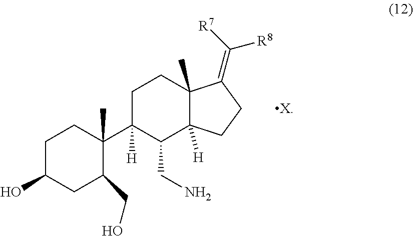

The present invention allows an alternative process to obtain the compound of formula (12) with fewer steps than known from conventional processes.

R⁷ and R⁸ are each independently selected as described above. Preferably R⁷ and R⁸ are independently hydrogen, halogen, linear or branched C₁-C₈ alkyl, C₁-C₈ alkoxy, C₆-C₁₀ aryloxy, C₃-C₁₀ cycloalkyl, C₅-C₁₀ aryl or C₇-C₁₀ arylalkyl. More preferably R⁷ and R⁸ are independently selected from the group consisting of hydrogen, fluorine, chlorine, methyl, ethyl, n-propyl, iso-propyl, n-butyl, iso-butyl, tert-butyl, methoxy, ethoxy, propoxy, butoxy, phenoxy, benzoxy, cyclopropyl, cyclobutyl, cycloheptyl, phenyl, naphthyl, tosyl, and benzyl. Even more preferably R⁷ and R⁸ are independently selected from the group consisting of hydrogen, fluorine, chlorine and methyl. Most preferably R⁷ and R⁸ are hydrogen.

X is an acid capable of forming a pharmaceutically acceptable salt with the compound of formula (11) to obtain compound of formula (12). Such acids can be any suitable acid known in the art. Examples of such acids are described in P. H. Stahl and C. G. Wermuth, editors, Handbook of Pharmaceutical Salts: Properties, Selection and Use, Weinheim/Zürich:Wiley-VCH/VHCA, 2011. Preferably, X is selected from the group consisting of any mineral acid and any organic mono- or di-acid. More preferably, X is selected from the group consisting of hydrochloric acid, hydrobromic acid, sulphuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Even more preferably X is selected from the group consisting of acetic acid, hydrochloric acid, hydrobromic acid, sulphuric acid, phosphoric acid, oxalic acid and tartaric acid. Even more preferably X is selected from the group consisting of acetic acid, oxalic acid and tartaric acid. Most preferably X is acetic acid.

The term second acid is equivalent to X and selected accordingly.

“Pharmaceutically acceptable salt” in the context of the present invention refers to acid addition salts.

Acid addition salts refer to those salts formed from compounds of the present invention and inorganic acids such as hydrochloric acid, hydrobromic acid, sulphuric acid, nitric acid, phosphoric acid and the like, and/or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

With the term “independently” is meant that each of the substituents may be chosen individually. That is, e.g. R⁷ and R⁸ can be different or the same; this means that in case of more than two substituents also one can be different from the others.

The substituents R¹ to R⁴³ of the present invention may be branched and/or substituted. Preferably R¹ to R⁴³ are unbranched. More preferably R¹ to R⁴³ are unbranched and unsubstituted.

The whole process or at least parts (e.g. single steps or even parts of single steps) of the process can either be performed in batch, semi-continuous or continuous production mode.

R³ may be any protective group for hydroxyl groups known in the art. Examples of suitable protective groups for hydroxyl groups are described in T. W. Greene, P. G. M. Wutts “Protective Groups in Organic Synthesis”, John Wiley & Sons, 5^{th} Ed., 2014, pages 17-374. Preferably, R³ is a linear or branched alkyl or alkenyl group having 1 to 8 carbon atoms, silyl, silane, siloxane, alkoxyalkyl, aryloxyalkyl, acyl, benzoyl, benzyl, methylthiomethyl, pivaloyl, pyranyl, or triarylalkyl. More preferably R³ is selected from the group consisting of methyl, ethyl, n-propyl, iso-propyl, n-butyl, iso-butyl, tert-butyl, trimethylsilyl (TMS), triethylsilyl (TES), tert.-butyldimethylsilyl (TBS/TBDMS), tert-butyldiphenylsilyl (TBDPS), triisopropylsilyl (TIPS), silane, tri-iso-propylsilyloxymethyl (TOM), methoxymethyl (MOM), ethoxyethyl (EE), benzyloxymethyl (BOM), 4-methoxybenzyl (PMB), acyl, benzoyl, benzyl, methylthiomethyl (MTM), pivaloyl, 2-tetrahydropyranyl (THP) and triphenylmethyl (Tr). More preferably R³ is selected from the group consisting of trimethylsilyl (TMS), triethylsilyl (TES), tert.-butyldimethylsilyl (TBS/TBDMS), tert-butyldiphenylsilyl (TBDPS), triisopropylsilyl (TIPS), silane, and tri-iso-propylsilyloxymethyl (TOM). Even more preferably R³ is selected from the group consisting of trimethylsilyl (TMS), triethylsilyl (TES), tert.-butyldimethylsilyl (TBS/TBDMS), tert-butyldiphenylsilyl (TBDPS) and triisopropylsilyl (TIPS). Most preferably R³ is tert.-butyldimethylsilyl (TBS/TBDMS).

R⁴² may be any protective group known in the art, in particular suitable to protect amino groups. Examples of suitable protective groups for amino groups are described in T. W. Greene, P. G. M. Wutts “Protective Groups in Organic Synthesis”, John Wiley & Sons, 5^{th} Ed., 2014, pages 895-1115. Preferably, R⁴² is a protective group for amino groups capable of forming a carbamate, an amide, a N-alkyl or N-aryl amine, or a N-hetero atom derivative with the compounds of the present invention. More preferably R⁴² is selected from the group consisting of alkoxycarbonyl, arylalkoxycarbonyl, formyl, acetyl, alkyl, aryl, arylalkyl and alkenyl. Even more preferably R⁴² is selected from the group consisting of methyl oxycarbonyl, ethyl oxycarbonyl, 9-flourenylmethyl oxycarbonyl, 1,1-dioxobenzo[b]thiophene-2-ylmethyl oxycarbonyl, 2-trimethylsilylethyl oxycarbonyl, 2-phenylethyl oxycarbonyl, 1-(1-adamantyl)-1-methylethyl oxycarbonyl, (2-pivaloylamino)-1,1-dimethylethyl oxycarbonyl, tert-butyl oxycarbonyl, 1-adamantyl oxycarbonyl, 2-adamantyl oxycarbonyl, 1-isopropylallyl oxycarbonyl, benzyl oxycarbonyl, formyl, acetyl, trifluoroacetyl, trichloroacetyl, phenylacetyl, benzyl, triphenylmethyl, p-tosyl, t-butyl, allyl and diphenylmethyl. Even more preferably R⁴² is selected from the group consisting of 9-flourenylmethyl oxycarbonyl, tert-butyl oxycarbonyl, formyl, acetyl, trifluoroacetyl, benzyl, triphenylmethyl and p-tosyl. Most preferably, R⁴² is tert-butyl oxycarbonyl.

“Heteroatom” may be a halogen, nitrogen, oxygen, phosphorous, silicon, boron or sulphur atom. Groups containing more than one heteroatom may contain different heteroatoms

X¹ represents a counter acid which together with the compounds of the present invention, in particular with compound of formula (9) may form a pharmaceutically acceptable salt to obtain compound of formula (10) and which generally may be selected as known in the art. Examples for the selection of X¹ are provided by P. H. Stahl and C. G. Wermuth, editors, Handbook of Pharmaceutical Salts: Properties, Selection and Use, Weinheim/Zürich:Wiley-VCHNHCA, 2011. Preferably, X¹ is selected from the group consisting of any mineral acid and any organic mono- or di-acid. More preferably, X¹ is selected from the group consisting of hydrochloric acid, hydrobromic acid, sulphuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Even more preferably X¹ is selected from the group consisting of acetic acid, benzenesulfonic acid, hydrochloric acid, hydrobromic acid, sulphuric acid, phosphoric acid, oxalic acid, tartaric acid and p-toluenesulfonic acid. Even more preferably X¹ is selected from the group consisting of benzenesulfonic acid and p-toluenesulfonic acid. Most preferably X¹ is p-toluenesulfonic acid.

The olefination reagent can be any suitable olefination reagent known in the art. The olefination reagent is generally a compound which is able to transform the keto group of compound of formula (10) into an alkene as present in compound of formula (11). Preferably, the olefination reagent is selected from the group consisting of a triphenylphosphonium ylide, a α-silyl carbanion, a stabilized phosphonate carbanion, an aryl sulfone, a sulfoxide, a Tebbe reagent, a Lombardo reagent and a Petasis reagent. More preferably, the olefination reagent is selected from the group consisting of a triphenylphosphonium methylide, α-trimethylsilyl acetate, α-trimethylsilyl aminoacetonitrile, methyldiethylphosphonoacetate, isopropyldiethylphosphoneacetate, tert.-butyldiethyl-phosphonoacetate, methyl phenyl sulfone, ethyl phenyl sulfone, benzothiazole methyl sulfone (BT-sulfone), 1-phenyl-1H-tetrazole methyl sulfone (PT-sulfone), a Tebbe reagent, a Lombardo reagent and a Petasis reagent. Even more preferably the olefination reagent is selected from the group consisting of triphenylphosphonium methylide, a Tebbe reagent, a Lombardo reagent and a Petasis reagent. Most preferably the olefination reagent is triphenylphosphonium methylide.

The triphenylphosphonium ylide may be generated in situ from methyltriphenylphosphonium bromide (Ph₃PMeBr) and potassium tert-butoxide (KOt-Bu).

In case the olefination reagent is a triphenylphosphonium ylide, a third base may be present to obtain the ylide from a phosphonium precursor which third base may be selected from organic or inorganic bases. Preferably, such third base is selected from the group consisting of potassium tert.-butoxide (KOt-Bu), potassium hydride with paraffin (KH(P)), sodium hydride (NaH), sodium hexamethyl disilylamide (NaHMDS), pyridine, triethylamine (NEt₃) lithiumdiisopropylamide (LDA) and sodium methoxide (NaOMe). More preferably such third base is selected from the group consisting of potassium tert.-butoxide (KOt-Bu), sodium hexamethyl disilylamide (NaHMDS), pyridine, triethylamine (NEt₃) lithiumdiisopropylamide (LDA) and sodium methoxide (NaOMe). Most preferably, such third base is potassium tert.-butoxide (KOt-Bu).

In case the olefination reagent is a α-silyl carbanion, an acid or a fourth base may be present in order to modify the stereoselectivity of the reaction. In case an acid is present, preferably the acid is selected from the group consisting of acetic acid (AcOH), sulphuric acid (H₂SO₄) and BF₃.OEt₂. More preferably the acid is selected from the group consisting of acetic acid and sulphuric acid. Most preferably the acid is sulphuric acid. In case a fourth base is present, preferably the fourth base is selected from the group consisting of sodium hydride (NaH), potassium hydride (KH) and potassium tert.-butoxide (KOt-Bu). More preferably the fourth base is selected from the group consisting of potassium hydride (KH) and potassium tert.-butoxide (KOt-Bu). Most preferably the fourth base is potassium tert.-butoxide (KOt-Bu).

In case the olefination reagent is an aryl sulfone, a metal containing fifth base may be present. Preferably such fifth base is a strong metal containing base. More preferably such fifth base is selected from the group consisting of sodium methoxide (NaOMe), sodium bis(trimethylsilyl)amide (NaHMDS) and lithium bis(trimethylsilyl) amide (LiHMDS). Most preferably such fifth base is sodium methoxide (NaOMe).

In case the olefination reagent is an aryl sulfone, the initially formed beta-oxido sulfone may be reduced by a second reducing agent. Such second reducing agent preferably is selected from the group consisting of Sml₂, Bu₃SnH, Li or Na in ammonia, Na₂S₂O4, Raney/Ni, Al(Hg) amalgam and LiAlH₄. More preferably such second reducing agent is selected from the group consisting of Sml₂, Li or Na in ammonia and Al(Hg) amalgam. Most preferably such second reducing agent is Sml₂.

Contacting a compound of formula (10) with an olefination reagent to obtain a compound of formula (11) may be performed in the presence of a solvent or mixture of solvents. Suitable solvents include water, organic solvents or mixtures of organic solvents or mixtures thereof. Preferably the solvent is selected from the group consisting of water, methanol, tetrahydrofuran (THF), toluene, xylene and benzene or mixtures thereof. More preferably the solvent is selected from the group consisting of methanol and tetrahydrofuran (THF) or mixtures thereof. Most preferably the solvent is tetrahydrofuran (THF).

Generally, the reaction temperature for producing a compound of formula (11) by contacting a compound of formula (10) with an olefination reagent ranges from −90° C. up to reflux of the solvent or solvent mixture. Preferably, the temperature is at least −80° C., more preferably at least −60° C., even more preferably at least −50° C., even more preferably at least −30° C., even more preferably at least −15° C., even more preferably at least −5° C., most preferably at least 0° C. Preferably, the temperature is at most 70° C., more preferably at most 60° C., even more preferably at most 45° C., and most preferably at most 30° C.

In one aspect of the invention the process further comprises a step of contacting a compound of formula (3)

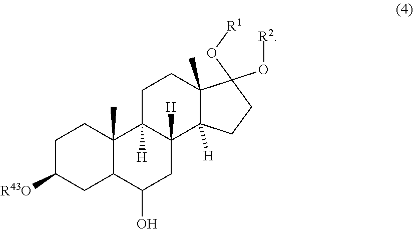

wherein R¹, R² and R³ are protective groups, with a second oxidizing agent to obtain a compound of formula (4)

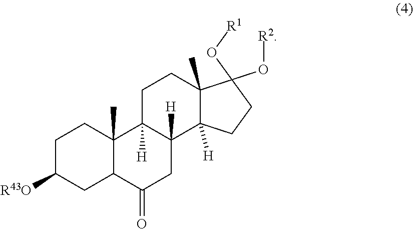

This step allows for the production of the compounds of formula (10) and formula (11) via various routes. This step renders any process for producing the compound of formula (11) to be distinctively different from the existing processes of the prior art, in particular to U.S. Pat. No. 6,046,185 and WO 2004/09210. A further advantage is that the compound of formula (4) can be isolated as a solid, which allows for an increased flexibility of the process, e.g. further processing at a different location.

R³ is a protective group as previously disclosed.

R¹ and R² are protective groups. Such protective groups are independently chosen and/or together form a cyclic protective group. Such protective groups may be any protective group known in the art, in particular suitable to protect hydroxyl groups. Examples of suitable protective groups for hydroxyl groups are described in T. W. Greene, P. G. M. Wutts “Protective Groups in Organic Synthesis”, John Wiley & Sons, 5^{th} Ed., 2014, pages 17-374. Preferably, R¹ and R² independently are linear or branched alkyl or alkenyl groups having 1 to 8 carbon atoms, silyl, silane, siloxane, alkoxyalkyl, aryloxyalkyl, acyl, benzoyl, benzyl, methylthiomethyl, pivaloyl, pyranyl, triarylalkyl, or together form a hydrocarbon group having the formula

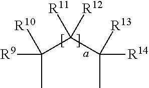

wherein a is 0 or 1 and R⁹, R¹⁰, R¹¹, R¹², R¹³ and R¹⁴ are independently hydrogen, alkynyl or linear, branched, or cyclic alkyl or alkenyl groups having 1 to 18 carbon atoms, or an aryl or alkylaryl group, wherein the alkyl, alkenyl, aryl, or alkylaryl groups include one or more additional functional groups such as halogen, tertiary amine, carbonate, ether, ester, carbonyl, urethane, imide, amide, sulfone, sulphonamide, mercapto, disulphide, phosphate, phosphonoxy, silane, or siliyl. More preferably R¹ and R² are independently selected from the group consisting of methyl, ethyl, n-propyl, iso-propyl, n-butyl, iso-butyl, tert-butyl, trimethylsilyl (TMS), triethylsilyl (TES), tert.-butyldimethylsilyl (TBS/TBDMS), tert-butyldiphenylsilyl (TBDPS), triisopropylsilyl (TIPS), silane, tri-iso-propylsilyloxymethyl (TOM), methoxymethyl (MOM), ethoxyethyl (EE), benzyloxymethyl (BOM), 4-methoxybenzyl (PMB), acyl, benzoyl, benzyl, methylthiomethyl (MTM), pivaloyl, 2-tetrahydropyranyl (THP) and triphenylmethyl (Tr), or together form a hydrocarbon group having the formula

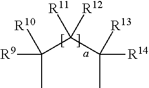

wherein a is 0 or 1 and R⁹, R¹⁰, R¹¹, R¹², R¹³ and R¹⁴ are independently hydrogen, ethinyl, 1-propinyl, 3-propinyl, 1-butin-1-yl, 2-butin-1-yl, 3-butin-1-yl, methyl, ethyl, n-propyl, iso-propyl, n-butyl, iso-butyl, tert-butyl, pentane, isopentane, neopentane, cyclopropane, cyclobutane, cyclopentane, cyclohexane, vinyl, allyl, crotyl, cyclopentadienyl, phenyl or benzyl. Even more preferably R¹ and R² form a hydrocarbon group having the formula

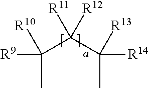

wherein a is 0 or 1 and R⁹, R¹⁰, R¹¹, R¹², R¹³ and R¹⁴ are independently hydrogen, ethinyl, 1-propinyl, 3-propinyl, 1-butin-1-yl, 2-butin-1-yl, 3-butin-1-yl, methyl, ethyl, n-propyl, iso-propyl, n-butyl, iso-butyl, tert-butyl, pentane, isopentane, neopentane, cyclopropane, cyclobutane, cyclopentane, cyclohexane, vinyl, allyl, crotyl, cyclopentadienyl, phenyl or benzyl. Even more preferably R¹ and R^{e}form a hydrocarbon group having the formula

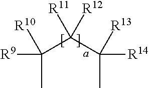

wherein a is 0 and R⁹, R¹⁰, R¹³ and R¹⁴ are independently hydrogen, methyl, ethyl, n-propyl, iso-propyl, n-butyl, iso-butyl, tert-butyl. Most preferably R¹ and R² form a hydrocarbon group having the formula

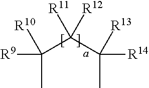

wherein a is 0 and R⁹, R¹⁰, R¹³ and R¹⁴ are independently hydrogen.

The second oxidizing agent is a member of the group of compounds which is able to transform a hydroxyl group into a keto group, in particular in compound of formula (3) of the invention. Preferably the second oxidizing agent of the invention is selected from the group consisting of dichromate, chromium trioxide (CrO₃), chromium trioxide derivatives, hypervalent iodines, oxalyl chloride, trifluoroacetic anhydride, metal alkoxides, silver(I) carbonate, activated dimethyl sulfoxide (DMSO) and tetrapropylammoniumperruthenat (TPAP). More preferably the second oxidizing agent of the invention is selected from the group consisting of potassium dichromate, pyridinium dichromate (PDC), chromium trioxide (CrO₃), CrO₃-amine reagents, 2-iodoxybenzoic acid, Dess-Martin periodinane (DMP), oxalyl chloride, trifluoroacetic anhydride, activated dimethyl sulfoxide (DMSO), aluminium isopropoxide (Al(i-PrO)₃), potassium tert-butoxide (KOt-Bu), silver(I) carbonate and tetrapropylammoniumperruthenat (TRAP). Even more preferably the second oxidizing agent of the invention is selected from the group consisting of potassium dichromate, pyridinium dichromate (PDC), chromium trioxide (CrO₃), pyridinium chlorochromate, 2-iodoxybenzoic acid, Dess-Martin periodinane (DMP) and activated dimethyl sulfoxide (DMSO). Even more preferably the second oxidizing agent of the present invention is selected from the group consisting of 2-iodobenzoic acid and Dess-Martin periodinane (DMP). Most preferably the second oxidizing agent of the present invention is 2-iodobenzoic acid.

Suitable means to activate dimethyl sulfoxide include the sulfur trioxide pyridine complex (pyridine-50₃ complex) in the presence of trimethylamine (TEA), N-chlorosuccinimide (NCS) and dimethylsulfide (DMS) in the presence of trimethylamine (TEA) or dicyclohexyl carbodiimide (DCC) and catalytic amounts of (moderately acidic) acid (e.g. anhydrous phosphoric acid (H₃PO₄) or dichloroacetic acid (Cl₂CCO₂H) or pyridiniumsalts of strong acids (e.g pyr-HCl, pyr-TFA).

The transformation of compound of formula (3) with a second oxidizing agent to obtain a compound of formula (4) may be performed in the presence of a solvent or a mixture of solvents. Suitable solvents include ethyl acetate (EtOAc), dimethyl sulfoxide (DMSO), dichloromethane (DCM), acetone, toluene, benzene, acetonitrile (MeCN), ionic liquids or tetrahydrofuran (THF). Preferably the solvent is selected from the group consisting of ethyl acetate (EtOAc), dimethyl sulfoxide (DMSO), dichloromethane (DCM) and toluene or mixtures thereof. Even more preferably the solvent is selected from the group consisting of ethyl acetate (EtOAc) and dimethyl sulfoxide (DMSO) or mixtures thereof. Even more preferably the solvent is a mixture of ethyl acetate (EtOAc) and dimethyl sulfoxide (DMSO). Most preferably the solvent is dimethyl sulfoxide (DMSO).

Generally, the reaction temperature for producing a compound of formula (4) by contacting a compound of formula (3) with a second oxidizing agent ranges from −20° C. up to reflux of the solvent or solvent mixture. Preferably, the reaction temperature is at least −10° C., more preferably at least 0° C., even more preferably at least 10° C. and most preferably at least 20° C. Preferably, the reaction temperature is at most 70° C., more preferably at most 60° C., even more preferably at most 45° C. and most preferably at most 30° C.

The invention further pertains to a process for preparing a compound of formula (4)

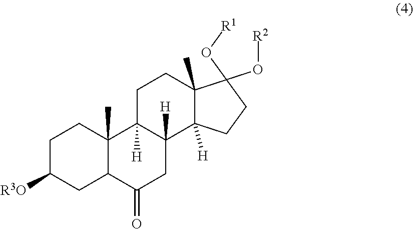

wherein R¹, R² and R³ are protective groups, comprising the step of contacting a compound of formula (3)

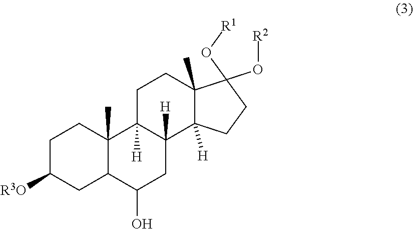

with a second oxidizing agent to obtain the compound of formula (4).
R¹, R² and R³ are selected as described above.

The invention further pertains to the compound of formula (4)

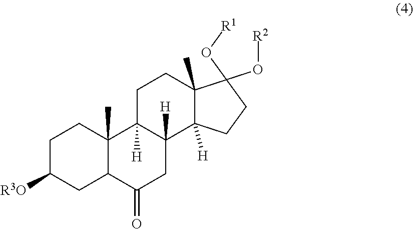

wherein R¹, R² and R³ are protective groups.

In another aspect of the invention the process further comprises
a) converting the compound of formula (4) to a compound of formula (7)

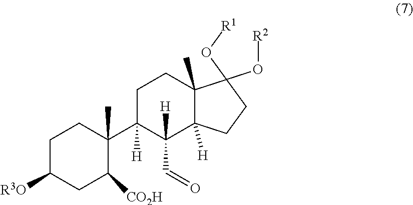

comprising the steps selected from:
   i) contacting the compound of formula (4) with a second base and a third protective reagent to form a compound of formula (5)

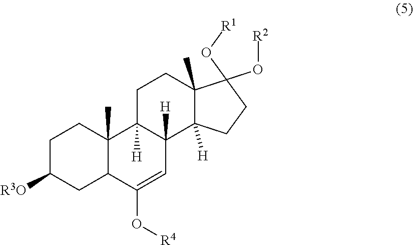

wherein R⁴ is a protective group, and
contacting the compound of formula (5) with a third oxidizing agent and optionally with a fourth protective reagent to obtain a compound of formula (6)

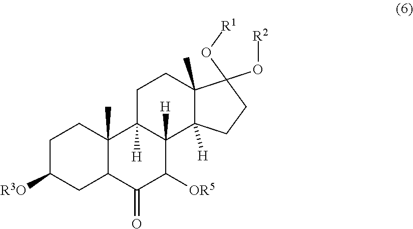

wherein R⁵ is H or a protective group; and
contacting the compound of formula (6) with a fourth oxidizing agent to obtain the compound of formula (7); or
   ii) contacting the compound of formula (4) with a second base and a third oxidizing agent and optionally with a fourth protective reagent to obtain a compound of formula (6), wherein the molar ratio of the third oxidizing agent and the compound of formula (4) is at most 1.5; and contacting a compound of formula (6) with a fourth oxidizing agent to obtain the compound of formula (7); or
   iii) contacting the compound of formula (4) with a second base and a third oxidizing agent and optionally with a fourth protective reagent to obtain the compound of formula (7), wherein the molar ratio of the third oxidizing agent and the compound of formula (4) is at least 1.5;

wherein the process further comprises the steps of:
a) contacting the compound of formula (7) with a compound of formula (7a)

NH₂—R⁶ (7a)

wherein R⁶ is either H or —OR²², wherein R²² is either H or a protective group, to obtain a compound of formula (8)

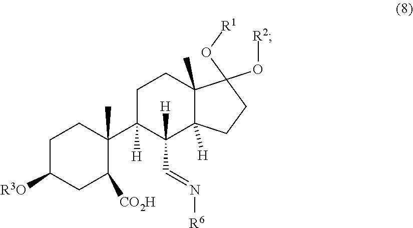

and
b) contacting the compound of formula (8) with a reducing agent to obtain the compound of formula (9).

Steps i), ii), iii) are alternative processes to convert compound of formula (4) to compound of formula (7). The compounds of formula (4), (5), (6), (7), (8) and (9) are novel.

The compounds of formula (4), (5), (6), (7), (8) and (9) may provide novel intermediates in the process of the invention. The compounds of formula (4), (5), (6), (7), (8) and (9) may be isolated as solids.

R¹, R² and R³ are protective groups as described above.

R⁴ may be any protective group known in the art, in particular suitable to protect hydroxyl groups. Examples of suitable protective groups for hydroxyl groups are described in T. W. Greene, P. G. M. Wutts “Protective Groups in Organic Synthesis”, John Wiley & Sons, 5^{th} Ed., 2014, pages 17-374. Preferably R⁴ is a linear or branched alkyl or alkenyl group having 1 to 8 carbon atoms, silyl, silane, siloxane, alkoxyalkyl, aryloxyalkyl, acyl, benzoyl, benzyl, methylthiomethyl, pivaloyl, pyranyl, or triarylalkyl. More preferably R⁴ is selected from the group consisting of methyl, ethyl, n-propyl, iso-propyl, n-butyl, iso-butyl, tert-butyl, trimethylsilyl (TMS), triethylsilyl (TES), tert.-butyldimethylsilyl (TBS/TBDMS), tert-butyldiphenylsilyl (TBDPS), triisopropylsilyl (TIPS), silane, tri-iso-propylsilyloxymethyl (TOM), methoxymethyl (MOM), ethoxyethyl (EE), benzyloxymethyl (BOM), 4-methoxybenzyl (PMB), acyl, benzoyl, benzyl, methylthiomethyl (MTM), pivaloyl, 2-tetrahydropyranyl (THP) and triphenylmethyl (Tr). More preferably R⁴ is selected from the group consisting of trimethylsilyl (TMS), triethylsilyl (TES), tert.-butyldimethylsilyl (TBS/TBDMS), tert-butyldiphenylsilyl (TBDPS), triisopropylsilyl (TIPS), silane and tri-iso-propylsilyloxymethyl (TOM). Even more preferably R⁴ is selected from the group consisting of trimethylsilyl (TMS), triethylsilyl (TES), tert.-butyldimethylsilyl (TBS/TBDMS), tert-butyldiphenylsilyl (TBDPS) and triisopropylsilyl (TIPS). Most preferably R⁴ is tert.-butyldimethylsilyl(TBS/TBDMS).

Optionally the hydroxyl group in alpha position to the keto group in compound of formula (6) may be protected by a protecting group for hydroxyl groups.

R⁵ may be H or any protective group known in the art, in particular suitable to protect hydroxyl groups. Examples of suitable protective groups for hydroxyl groups are described in T. W. Greene, P. G. M. Wutts “Protective Groups in Organic Synthesis”, John Wiley & Sons, 5^{th} Ed., 2014, pages 17-374. Preferably R⁵ is H, a linear or branched alkyl or alkenyl group having 1 to 8 carbon atoms, silyl, silane, siloxane, alkoxyalkyl, aryloxyalkyl, acyl, benzoyl, benzyl, methylthiomethyl, pivaloyl, pyranyl, or triarylalkyl. More preferably R⁵ is selected from the group consisting of H, methyl, ethyl, n-propyl, iso-propyl, n-butyl, iso-butyl, tert-butyl, trimethylsilyl (TMS), triethylsilyl (TES), tert.-butyldimethylsilyl (TBS/TBDMS), tert-butyldiphenylsilyl (TBDPS), triisopropylsilyl (TIPS), silane, tri-iso-propylsilyloxymethyl (TOM), methoxymethyl (MOM), ethoxyethyl (EE), benzyloxymethyl (BOM), 4-methoxybenzyl (PMB), acyl, benzoyl, benzyl, methylthiomethyl (MTM), pivaloyl, 2-tetrahydropyranyl (THP) and triphenylmethyl (Tr). More preferably R⁵ is selected from the group consisting of H, trimethylsilyl (TMS), triethylsilyl (TES), tert.-butyldimethylsilyl (TBS/TBDMS), tert-butyldiphenylsilyl (TBDPS), triisopropylsilyl (TIPS), silane, and tri-iso-propylsilyloxymethyl (TOM). Even more preferably R⁵ is selected from the group consisting of H, trimethylsilyl (TMS), triethylsilyl (TES), tert.-butyldimethylsilyl (TBS/TBDMS), tert-butyldiphenylsilyl (TBDPS) and triisopropylsilyl (TIPS). Most preferably R⁵ is H.

The process further comprises contacting a compound of formula (7) with a compound of formula (7a) to obtain a compound of formula (8).

Compound of formula (7a) is a member of the group of compounds which is able to transform the aldehyde moiety of compound of formula (7) into an imine or more specifically into an oxime as present in compound of formula (8).

Preferably R⁶ is —OR²².

R²² may be H or any protective group known in the art, in particular suitable to protect hydroxyl groups. Examples of suitable protective groups for R²² are described in T. W. Greene, P. G. M. Wutts “Protective Groups in Organic Synthesis”, John Wiley & Sons, 5th Ed., 2014, pages 17-374. Preferably R²² is selected from the group consisting of H, a linear or branched alkyl or alkenyl group having 1 to 8 carbon atoms, silyl, silane, siloxane, alkoxyalkyl, aryloxyalkyl, acyl, benzoyl, benzyl, methylthiomethyl, pivaloyl, pyranyl, and triarylalkyl. More preferably R²² is selected from the group consisting of H, methyl, ethyl, n-propyl, iso-propyl, n-butyl, iso-butyl, tert-butyl, trimethylsilyl (TMS), triethylsilyl (TES), tert.-butyldimethylsilyl (TBS/TBDMS), tert-butyldiphenylsilyl (TBDPS), triisopropylsilyl (TIPS), silane, tri-iso-propylsilyloxymethyl (TOM), methoxymethyl (MOM), ethoxyethyl (EE), benzyloxymethyl (BOM), 4-methoxybenzyl (PMB), acyl, benzoyl, benzyl, methylthiomethyl (MTM), pivaloyl, 2-tetrahydropyranyl (THP) and triphenylmethyl (Tr). Even more preferably R²² is selected from the group consisting of H, trimethylsilyl (TMS), triethylsilyl (TES), tert.-butyldimethylsilyl (TBS/TBDMS), tert-butyldiphenylsilyl (TBDPS), triisopropylsilyl (TIPS), silane and tri-iso-propylsilyloxymethyl (TOM). Even more preferably R²² is selected from the group consisting of trimethylsilyl (TMS), triethylsilyl (TES), tert.-butyldimethylsilyl (TBS/TBDMS), tert-butyldiphenylsilyl (TBDPS) and triisopropylsilyl (TIPS). Most preferably R²² is H.

Compound of formula (7a) is preferably selected from the group consisting of ammonia (NH₃), hydroxylamine, derivatives of hydroxylamine (e.g. hydroxylamine-O-sulfonic acid (H₂NOSO₃H), HON(SO₃Na)₂, NH₂OH.HCl, alkoxyamine or aralkyoxyamine. More preferably compound of formula (7a) is selected from the group consisting of ammonia (NH₃), hydroxylamine, hydroxylamine-O-sulfonic acid, NH₂OH.HCl, linear or branched C₁-C₆ alkoxylamine or C₇-C₁₃ aralkoxyamine. Even more preferably compound of formula (7a) is selected from the group consisting of ammonia (NH₃), hydroxylamine, hydroxylamine-O-sulfonic acid, NH₂OH.HCl, O-methylhydroxylamine or O-benzylhydroxylamine. Most preferably compound of formula (7a) is hydroxylamine.

The second base is selected from the group of compounds which is able to stabilize an enol moiety derived from the keto group, in particular in compound of formula (4). The second base of the present invention includes any strong bulky organic base. Preferably the second base is selected from the group sodium tert.-butoxide (NaO-tBu), potassium tert.-butoxide (KO-tBu), lithium di-isopropyl amide (LDA), triethylamine, 2,6-dimethylpyridine, 1, 5-diazabicyclo[4.3.0]non-5-ene (DBN), 1,8-diazabicyclo[5.4.0]undec-7-ene (DBU) and lithium-bis(trimethylsilyl)amide (LHMDS). More preferably the second base is selected from the group lithium di-isopropyl amide (LDA), trimethylamine, dimethylpyridine and lithium-bis(trimethylsilyl)amide (LHMDS). Most preferably the second base is lithium di-isopropyl amide (LDA).

The third protective reagent is a member of the group of compounds which is able to form a protective group for hydroxyl groups and is generally selected as known in the art. Examples of suitable third protective reagents to obtain protective groups for hydroxyl groups are described in T. W. Greene, P. G. M. Wutts “Protective Groups in Organic Synthesis”, John Wiley & Sons, 5^{th} Ed., 2014, pages 17-374. Preferably, the third protective reagent is selected from the group consisting of alkylsulfate (CH₃SO₄), alkyliodide (CH₃1), diazoalkane (CH₂N₂), dialkylhydrogenphosphite ((MeO)₂POH), isobutylene (C₄H₈), allylbromide, trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl), tri-iso-propylsilyloxymethylchloride, chloromethylmethylether (CMME), bromomethylmethylether, iodomethylmethylether, dimethoxymethane (CH₂(OMe)₂), diethoxyethane (CH₂(OEt)₂ (Phenyldimethylsilyl)methoxymethylchloride (SMOMCl), ethylvinylether, 2-(chloromethoxy)-2-methylpropane (t-BuOCH₂C1), ((tert-butoxymethyl)sulfonyl)benzene (t-BuOCH₂SO₂Ph), 4-pentenyloxymethylchloride (POMCl), 2-methoxyethoxymethyl chloride (MEMCl), 2,2, 2-trichloroethoxymethoxychloride, 2,2,2-trichloroethoxymethoxybromide, (4-methoxyphenoxy)methylchloride (p-AOMCl), p-methoxybenzyloxymethylchloride (PMBMCl), benzyloxymethylchloride (PhCH₂OCH₂OCl), p-methoxyphenol, p-methoxybenzyl 2,2, 2-trichloroacetimidate, guaiacolmethylchloride (2-MeOC₆H₄OCH₂C1), acetic anhydride (Ac₂O), benzoylchloride (BzCl), benzylbromide, benzylchloride, iodomethylmethylsulfane (CH₃SHC₂l), chloromethylmethylsulfane (CH₃SHC₂Cl), dimethylsulfide (CH₃SCH₃), oxalylchloride (COCl)₂, pivaloyl chloride (PivCl), 3,4-dihydropyran and tritylchloride. More preferably, the third protective reagent is selected from the group consisting of trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl) and tri-iso-propylsilyloxymethylchloride. Even more preferably the third protective reagent is selected from the group consisting of trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl) and t-butyldiphenylsilylchloride (TBDPSCl). Most preferably, the third protective reagent of the present invention is t-butyldimethylsilylchloride (TBDMSCl).

The fourth protective reagent is a member of the group of compounds which is able to form a protective group for hydroxyl groups and can be any suitable protective group known in the art. Examples of suitable fourth protective reagents to obtain protective groups for hydroxyl groups are described in T. W. Greene, P. G. M. Wutts “Protective Groups in Organic Synthesis”, John Wiley & Sons, 5^{th} Ed., 2014, pages 17-374. Preferably the fourth protective reagent is selected from the group consisting of alkylsulfate (CH₃SO₄), alkyliodide (CH₃1), diazoalkane (CH₂N₂), dialkylhydrogenphosphite ((MeO)₂POH), isobutylene (C₄H₈), allylbromide, trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl), tri-iso-propylsilyloxymethylchloride, chloromethylmethylether (CMME), bromomethylmethylether, iodomethylmethylether, dimethoxymethane (CH₂(OMe)₂), diethoxyethane (CH₂(OEt)₂ (Phenyldimethylsilyl)methoxymethylchloride (SMOMCl), ethylvinylether, 2-(chloromethoxy)-2-methylpropane (t-BuOCH₂Cl), ((tert-butoxymethyl)sulfonyl)benzene (t-BuOCH₂SO₂Ph), 4-pentenyloxymethylchloride (POMCl), 2-methoxyethoxymethyl chloride (MEMCl), 2,2, 2-trichloroethoxymethoxychloride, 2,2,2-trichloroethoxymethoxybromide, (4-methoxyphenoxy)methylchloride (p-AOMCl), p-methoxybenzyloxymethylchloride (PMBMCl), benzyloxymethylchloride (PhCH₂OCH₂OCl), p-methoxyphenol, p-methoxybenzyl 2,2, 2-trichloroacetimidate, guaiacolmethylchloride (2-MeOC₆H₄OCH₂Cl), acetic anhydride (Ac₂O), benzoylchloride (BzCl), benzylbromide, benzylchloride, iodomethylmethylsulfane (CH₃SHC₂l), chloromethylmethylsulfane (CH₃SHC₂Cl), dimethylsulfide (CH₃SCH₃), oxalylchloride (COCl)₂, pivaloyl chloride (PivCl), 3,4-dihydropyran or tritylchloride. More preferably, the fourth protective reagent of the present invention is selected from the group consisting of trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl), or tri-iso-propylsilyloxymethylchloride. Even more preferably the fourth protective reagent in the present invention is selected from the group consisting of trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl). Most preferably, the fourth protective reagent of the present invention is t-butyldimethylsilylchloride (TBDMSCl).

The third oxidizing agent is any sutable oxidizing agent capable of either transforming a keto group as present in compound of formula (4) or an enol moiety as present in compound of formula (5) to the corresponding secondary alpha-hydroxy-ketone moiety of compound of formula (6). Optionally, the third oxidizing agent is a is an oxidizing agent furthermore capable of transforming the alpha-hydroxy-ketone moiety of compound of formula (6) into two separated functional groups, i.e. an aldehyde and a carboxylic acid, linked to different cyclic moieties as present in compound of formula (7). Preferably, the third oxidizing agent of the invention is selected from the group consisting of potassium peroxymonosulfate (KHSO₅), oxaziridine, ozone, peroxyacid, hydrogen peroxide and hypofluorous acid (HOF). More preferably the third oxidazing agent of the invention is selected from the group consisting of potassium peroxymonosulfate (KHSO5), sulfonyloaziridine or Davis' chiral oxaziridine, ozone, a peroxyacid, hydrogen peroxide or hypofluorous acid (HOF). Even more preferably, the third oxidizing agent of the invention is selected from the group consisting of potassium peroxymonosulfate (KHSO₅), peroxyacid and hydrogen peroxide. Even more preferably the third oxidizing agent of the invention is selected from the group consisting of potassium peroxymonosulfate (KHSO₅), meta-chloroperoxybenzoic acid (mCPBA) and hydrogen peroxide. Most preferably, the third oxiziding agent of the invention is meta-chloroperoxybenzoic acid (mCPBA).

The fourth oxidizing agent is an oxidizing agent capable of transforming the alpha-hydroxy-ketone moiety of compound of formula (6) into two separated functional groups, i.e. an aldehyde and a carboxylic acid, linked to separate cyclic moieties as present in compound of formula (7). Examples of the fourth oxidizing agent of the present invention include percarbonates, periodates, lead tetraacetate (LTA), osmium complexes, bismuthate, bismuth(III) carboxylates, iodo triacetate, manganic pyrophosphate, manganese dioxide, potassium peroxymonosulfate (KHSO₅), calcium hypochlorite, basic hydrogen peroxide, methylrhenium trioxide, chromium(VI) reagents, ceric ammonium nitrate and vanadium-based heteropolyacids (HPA). Preferably, the fourth oxidizing agent of the invention is selected from the group consisting of sodium percarbonate, sodium metaperiodate (NalO₄), lead tetraacetate (LTA), sodium bismuthate (NaBiO₃), bismuth(III)mandelate, iodo triacetate, manganic pyrophosphate, manganese dioxide, potassium peroxymonosulfate (KHSO₅) and calcium hypochlorite. More preferably the fourth oxidizing agent of the invention is selected from the group consisting of sodium percarbonate, sodium metaperiodate (NalO₄), lead tetraacetate (LTA) and potassium peroxymonosulfate (KHSO₅). Even more preferably, the fourth oxidizing agent of the invention is selected from the group consisting of sodium metaperiodate and lead tetraacetate (LTA). Most preferably, the fourth oxidizing agent of the invention is sodium metaperiodate (NalO₄).

“Heteropolyacid” generally refers to an acid made up of a particular combination of hydrogen and oxygen with certain metals and non-metals. To qualify as a heteropoly acid, the compound must typically contain a metal such as tungsten, molybdenum or vanadium, termed the addenda atom, oxygen, an element generally from the p-block of the periodic table, such as silicon, phosphorus or arsenic, termed the hetero atom and acidic hydrogen atoms. The metal addenda atoms linked by oxygen atoms typically form a cluster with the hetero-atom inside bonded via oxygen atoms. Examples with more than one type of metal addenda atom in the cluster are well known.

Contacting the compound of formula (4) with a second base and a third protective reagent to form a compound of formula (5) may be performed in the presence of a solvent or a mixture of solvents. Suitable solvents include anhydrous tetrahydrofuran (THF), 2-methyltetrahydrofuran MeTHF, ether, dioxane, dimethoxyethane (DME), heptane, toluene, xylene or benzene. Preferably the solvent is selected from the group consisting of tetrahydrofuran (THF), diethyl ether, 1,4-dioxane, heptane and ethylbenzene or mixtures thereof. More preferably the solvent is selected from the group consisting of tetrahydrofuran (THF), diethyl ether, heptane and ethylbenzene or mixtures thereof. Most preferably the solvent is tetrahydrofuran (THF).

For contacting the compound of formula (4) with a second base and a third protective reagent to form a compound of formula (5) the molar ratio of the third protective reagent and compound of formula (4) is typically at least 0.8, preferably at least 0.9, and most preferably at least 1, and generally at most 2, preferably at most 1.8, even more preferably at most 1.6 and most preferably at most 1.5.

A suitable temperature for contacting the compound of formula (4) with a second base and a third protective reagent to form a compound of formula (5) is preferably at least −85° C., more preferably at least −80° C., even more preferably at least −75° C. and most preferably at least −70° C. A suitable temperature at step i. is preferably at most 50° C., more preferably at most 40° C., even more preferably at most 30° C. and most preferably at most 25° C.

Solvents, suitable for contacting the compound of formula (4) with a second base and a third oxidizing agent disclosed in step iii. include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably the solvent is selected from the group consisting of tetrahydrofuran (THF), dichloromethane (DCM) and toluene or mixtures thereof. Most preferably the solvent is tetrahydrofuran (THF).

Solvents, suitable for contacting the compound of formula (5) with a third oxidizing agent as disclosed in step i. or ii. include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably, the solvent is selected from the group consisting of tetrahydrofuran (THF), dichloromethane (DCM) and toluene or mixtures thereof. Most preferably, the solvent is dichloromethane (DCM).

For contacting compound of formula (5) with a third oxidizing agent as disclosed in step i. the molar ratio of the third oxidizing agent and compound of formula (5) is typically at least 0.8, preferably at least 0.9, and most preferably at least 1, and generally at most 2, preferably at most 1.8, even more preferably at most 1.6, even more preferably at most 1.4 and most preferably at most 1.3.

For contacting the compound of formula (4) with a third oxidizing agent as disclosed in step ii. the molar ratio of the third oxidizing agent and compound of formula (4) is typically at least 0.8, preferably at least 0.9, and most preferably at least 1, and generally at most 1.5, preferably at most 1.4 and most preferably at most 1.3.

For contacting the compound of formula (4) with a third oxidizing agent as disclosed in step iii. the molar ratio of the third oxidizing agent and compound of formula (4) is typically at least 1.5, preferably at least 1.7, and most preferably at least 2, and generally at most 3, preferably at most 2.8, even more preferably at most 2.6 and most preferably at most 2.5.

A suitable temperature for contacting the compound of formula (4) with a base and a third oxidizing agent or contacting the compound of formula (5) with a third oxidizing agent is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

In case R⁵ in compound of formula (6) shall be a protective group, a fourth protective reagent may be added to the compound obtained from either contacting a compound of formula (4) with a second base and a third oxidizing agent or contacting a compound of formula (5) with a third oxidizing agent. The molar ratio of the fourth protective reagent and the compound obtained from either contacting a compound of formula (4) with a second base and a third oxidizing agent or contacting a compound of formula (5) with a third oxidizing agent of formula (6) is typically at least 0.8, preferably at least 0.9, and most preferably at least 1, and generally at most 2, preferably at most 1.8, even more preferably at most 1.6 and most preferably at most 1.5.

In steps i. and ii. of the invention a compound of formula (6) is contacted with a fourth oxidizing agent to obtain a compound of formula (7).

The fourth oxidizing agent is a member of the group of compounds which is able to transform the alpha-hydroxy-ketone moiety of compound of formula (6) into two separated functional groups, i.e. an aldehyde and a carboxylic acid, linked to separate cyclic moieties as present in compound of formula (7).

Suitable fourth oxidizing agents of the present invention include percarbonates, perjodates, lead tetraacetate (LTA), osmium complexes, bismuthate, bismuth(III) carboxylates, iodo triacetate, manganic pyrophosphate, manganese dioxide, potassium peroxymonosulfate (KHSO₅), calcium hypochlorite, basic hydrogen peroxide, methylrhenium trioxide, chromium(VI) reagents, ceric ammonium nitrate and vanadium-based heteropolyacids (HPA). Preferably, the fourth oxidizing agent of the invention is selected from the group consisting of sodium percarbonate, sodium metaperiodate (NalO₄), lead tetraacetate (LTA), sodium bismuthate (NaBiO₃), bismuth(III)mandelate, iodo triacetate, manganic pyrophosphate, manganese dioxide, potassium peroxymonosulfate (KHSO₅) and calcium hypochlorite, ore preferably from the group consisting of sodium percarbonate, sodium metaperiodate (NalO₄), lead tetraacetate (LTA) and potassium peroxymonosulfate (KHSO₅), even more preferably from the group consisting of sodium metaperiodate and lead tetraacetate (LTA). Most preferably, the fourth oxidizing agent of the invention is sodium metaperiodate (NalO₄).

Contacting compound of formula (6) with a fourth oxidizing agent to obtain a compound of formula (7) may be performed in the presence or absence of a solvent or mixture of solvents.

Solvents, suitable for contacting compound of formula (6) with a fourth oxidizing agent to obtain a compound of formula (7) include organic solvents or mixtures of organic solvents with water. Preferably, the solvent suitable for contacting compound of formula (6) with a fourth oxidizing agent to obtain a compound of formula (7) are selected from the group consisting of methanol (MeOH), dimethyl sulfoxide (DMSO), tetrahydrofuran (THF), dichloromethane (DCM), chloroform (CHCl₃), toluene, xylene and benzene or mixtures thereof. More preferably, the solvent is selected from the group consisting of methanol (MeOH) and dimethyl sulfoxide (DMSO) or mixtures thereof. Most preferably, the solvent is methanol (MeOH).

For contacting compound of formula (6) with a fourth oxidizing agent to obtain a compound of formula (7) the molar ratio of the fourth oxidizing agent and compound of formula (6) is typically between 1 and 3. Preferably, for contacting compound of formula (6) with a fourth oxidizing agent to obtain a compound of formula (7) the molar ratio of the fourth oxidizing agent and compound of formula (6) is preferably at least 1.6, more preferably at least 1.8, most preferably at least 2, and preferably at most 2.8, more preferably at most 2.5 and most preferably at most 2.3.

A suitable temperature for contacting compound of formula (6) with a fourth oxidizing agent to obtain a compound of formula (7) is preferably at least 0° C., more preferably at least 10° C., even more preferably at least 15° C. and most preferably at least 20° C. A suitable temperature for contacting compound of formula (6) with a fourth oxidizing agent to obtain a compound of formula (7) is preferably at most 60° C., more preferably at most 50° C., even more preferably at most 45° C. and most preferably at most 30° C.

The step of contacting the compound of formula (7) with a compound of formula (7a) to obtain a compound of formula (8) may be performed in the presence of a solvent or mixture of solvents. Suitable solvents include water, organic solvents or mixtures of organic solvents with water. Preferably, the solvent in the step of contacting the compound of formula (7) with a compound of formula (7a) to obtain a compound of formula (8) is selected from the group consisting of water, methanol, ethanol, 1-butanol, 2-butanol, 1,2-dimethoxy-ethane (DME), tetrahydrofuran (THF) and 1,4-dioxane. More preferably, the solvent is selected from the group consisting of water, methanol, ethanol and tetrahydrofuran (THF) or mixtures thereof. Most preferably, the solvent is tetrahydrofuran (THF).

The molar ratio of compound of formula (7) and compound of formula (7a) when contacting compound of formula (7) with compound of formula (7a) to obtain a compound of formula (8) is typically at least 0.8, preferably at least 0.9, and most preferably at least 1, and generally at most 1.5, preferably at most 1.4 and most preferably at most 1.3.

The temperature at the step of contacting compound of formula (7) with compound of formula (7a) to obtain a compound of formula (8) is preferably at least 0° C., more preferably at least 10° C., even more preferably at least 15° C. and most preferably at least 20° C. The temperature at the step of contacting compound of formula (7) with compound of formula (7a) to obtain a compound of formula (8) is preferably at most 90° C., more preferably at most 70° C., even more preferably at most 50° C. and most preferably at most 35° C.

The compound of formula (8) is contacted with a reducing agent to obtain the compound of formula (9).

The reducing agent is a member of the group of compounds which is able to transform the imino group of compound of formula (8) into an amine as present in compound of formula (8). Preferably, the reducing agent is selected from the group consisting of hydrogen or an ionic or a metallic hydride. More preferably, the reducing agent is selected from the group consisting of hydrogen, alane (AlH₃), lithium aluminium hydride (LiAlH₄), sodium hydride (NaH) and diisobutylaluminiumhydride (DIBAL-H), even more preferably selected from the group consisting of hydrogen, alane (AlH₃), lithium aluminium hydride (LiAlH₄) and diisobutylaluminiumhydride (DIBAL-H) and most preferably the reducing agent is alane (AlH₃).

In case hydrogen is selected as reducing agent, catalysts may be present as known in the art. Examples of such catalysts include Ni, Co, Pd, Pt, Ru, Rh, Os, Ir, Os and Fe.

The step of contacting compound of formula (8) with a reducing agent to obtain the compound of formula (9) may be performed at atmospheric or elevated pressure. Preferably, the transformation is performed at atmospheric pressure.

Solvents, suitable for the step of contacting compound of formula (8) with a reducing agent to obtain the compound of formula (9) include organic solvents or mixtures of organic solvents. Preferably, the solvent for the step of contacting compound of formula (8) with a reducing agent to obtain the compound of formula (9) is selected from the group consisting of 1,2-dimethoxy-ethane (DME), tetrahydrofuran (THF), chloroform (CHCl₃), toluene and benzene or mixtures thereof. More preferably, the solvent is selected from the group consisting of tetrahydrofuran (THF) and toluene or mixtures thereof. Most preferably, the solvent is tetrahydrofuran (THF).

The invention further pertains to a process comprising contacting the compound of formula (4)

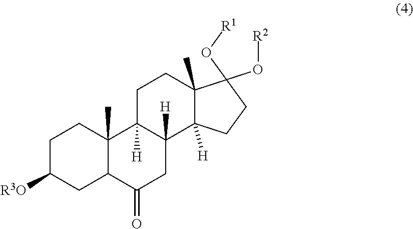

wherein R¹, R² and R³ are protective groups, with a second base and a third protective reagent to form a compound of formula (5)

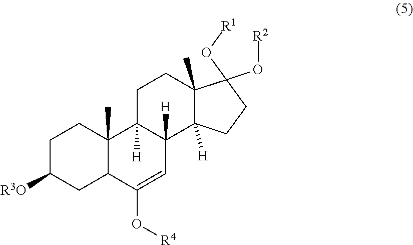

wherein R¹, R², R³ and R⁴ are protective groups.
R¹, R², R³, R⁴, the second base and the third protective reagent are selected as described above.

Contacting the compound of formula (4) with a second base and a third protective reagent to form a compound of formula (5) may be performed in the presence of a solvent or a mixture of solvents. Suitable solvents include anhydrous tetrahydrofuran (THF), 2-methyltetrahydrofuran MeTHF, ether, dioxane, dimethoxyethane (DME), heptane, toluene, xylene or benzene. Preferably, the solvent is selected from the group consisting of tetrahydrofuran (THF), diethyl ether, 1,4-dioxane, heptane and ethylbenzene or mixtures thereof. More preferably the solvent is selected from the group consisting of tetrahydrofuran (THF), diethyl ether, heptane and ethylbenzene or mixtures thereof. Most preferably, the solvent is tetrahydrofuran (THF).

For contacting the compound of formula (4) with a second base and a third protective reagent to form a compound of formula (5) the molar ratio of the third protective reagent and compound of formula (4) is typically at least 0.8, preferably at least 0.9, and most preferably at least 1, and generally at most 2, preferably at most 1.8, even more preferably at most 1.6 and most preferably at most 1.5.

A suitable temperature for contacting the compound of formula (4) with a second base and a third protective reagent to form a compound of formula (5) is preferably at least −85° C., more preferably at least −80° C., even more preferably at least −75° C. and most preferably at least −70° C. A suitable temperature at step i. is preferably at most 50° C., more preferably at most 40° C., even more preferably at most 30° C. and most preferably at most 25° C.

The invention further pertains to a process comprising contacting compound of formula (5)

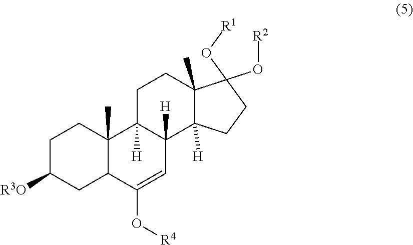

wherein R¹, R², R³ and R⁴ are protective groups, with a third oxidizing agent and optionally with a fourth protective reagent to obtain a compound of formula (6)

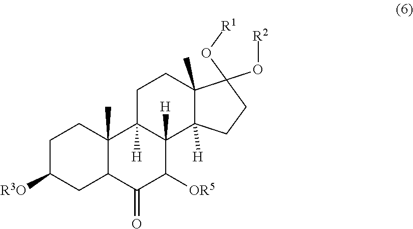

wherein R¹, R² and R³ are protective groups and R⁵ is H or a protective group.
R¹, R², R³, R⁵, the third oxidizing agent and the fourth protective reagent are as described above.

Solvents, suitable for contacting the compound of formula (5) with a third oxidizing agent to obtain a compound of formula (6) include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably, the solvent is selected from the group consisting of tetrahydrofuran (THF), dichloromethane (DCM) and toluene or mixtures thereof. Most preferably, the solvent is dichloromethane (DCM).

For contacting compound of formula (5) with a third oxidizing agent to obtain a compound of formula (6) the molar ratio of the third oxidizing agent and compound of formula (5) is typically at least 0.8, preferably at least 0.9, and most preferably at least 1, and generally at most 2, preferably at most 1.8, even more preferably at most 1.6, even more preferably at most 1.4 and most preferably at most 1.3.

A suitable temperature for contacting the compound of formula (5) with a third oxidizing agent to obtain a compound of formula (6) is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

In case R⁵ in compound of formula (6) shall be a protective group, a fourth protective reagent may be added to the compound obtained from contacting a compound of formula (5) with a third oxidizing agent. The molar ratio of the fourth protective reagent and the compound obtained from contacting a compound of formula (5) with a third oxidizing agent is typically at least 0.8, preferably at least 0.9, and most preferably at least 1, and generally at most 2, preferably at most 1.8, even more preferably at most 1.6 and most preferably at most 1.5.

The invention further pertains to a process comprising contacting compound of formula (6)

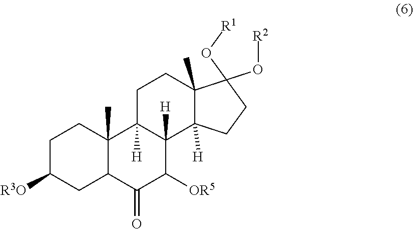

wherein R¹, R² and R³ are protective groups and R⁵ is H or a protective group, with a fourth oxidizing agent to obtain the compound of formula (7)

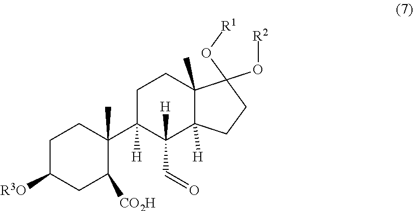

wherein R¹, R² and R³ are protective groups.
R¹, R², R³, and the fourth oxidizing agent are selected as described above.

Contacting compound of formula (6) with a fourth oxidizing agent to obtain a compound of formula (7) may be performed in the presence or absence of a solvent or mixture of solvents.

Solvents, suitable for contacting compound of formula (6) with a fourth oxidizing agent to obtain a compound of formula (7) include organic solvents or mixtures of organic solvents with water. Preferably, the solvent suitable for contacting compound of formula (6) with a fourth oxidizing agent to obtain a compound of formula (7) are selected from the group consisting of methanol (MeOH), dimethyl sulfoxide (DMSO), tetrahydrofuran (THF), dichloromethane (DCM), chloroform (CHCl₃), toluene, xylene and benzene or mixtures thereof. More preferably, the solvent is selected from the group consisting of methanol (MeOH) and dimethyl sulfoxide (DMSO) or mixtures thereof. Most preferably, the solvent is methanol (MeOH).

For contacting compound of formula (6) with a fourth oxidizing agent to obtain a compound of formula (7) the molar ratio of the fourth oxidizing agent and compound of formula (6) is typically between 1 and 3. Preferably, for contacting compound of formula (6) with a fourth oxidizing agent to obtain a compound of formula (7) the molar ratio of the fourth oxidizing agent and compound of formula (6) is preferably at least 1.6, more preferably at least 1.8, most preferably at least 2, and preferably at most 2.8, more preferably at most 2.5 and most preferably at most 2.3.

A suitable temperature for contacting compound of formula (6) with a fourth oxidizing agent to obtain a compound of formula (7) is preferably at least 0° C., more preferably at least 10° C., even more preferably at least 15° C. and most preferably at least 20° C. A suitable temperature for contacting compound of formula (6) with a fourth oxidizing agent to obtain a compound of formula (7) is preferably at most 60° C., more preferably at most 50° C., even more preferably at most 45° C. and most preferably at most 30° C.

The invention further pertains to a process comprising contacting the compound of formula (4)

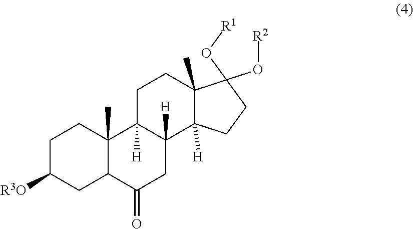

wherein R¹, R² and R³ are protective groups, with a second base and a third protective reagent to form a compound of formula (5)

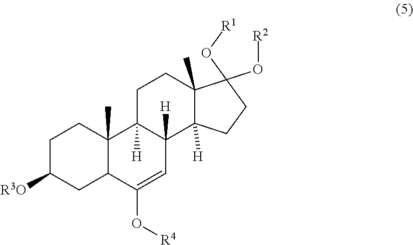

wherein R⁴ is a protective group, and
contacting the compound of formula (5) with a third oxidizing agent and optionally with a fourth protective reagent to obtain a compound of formula (6)

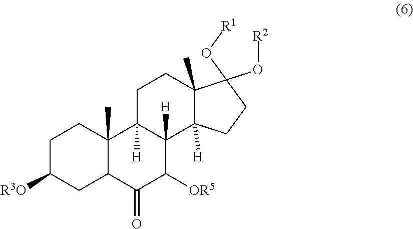

wherein R⁵ is H or a protective group; and
contacting the compound of formula (6) with a fourth oxidizing agent to obtain the compound of formula (7).

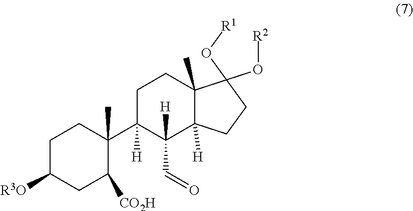

wherein R¹, R² and R³ are protective groups.
R¹, R², R³, R⁴, R⁵, the second base, the second protective reagent, the third oxidizing agent, the fourth protective reagent and the fourth oxidizing agent are selected as described above.

Contacting the compound of formula (4) with a second base and a third protective reagent to form a compound of formula (5) may be performed in the presence of a solvent or a mixture of solvents. Suitable solvents include anhydrous tetrahydrofuran (THF), 2-methyltetrahydrofuran MeTHF, ether, dioxane, dimethoxyethane (DME), heptane, toluene, xylene or benzene. Preferably, the solvent is selected from the group consisting of tetrahydrofuran (THF), diethyl ether, 1,4-dioxane, heptane and ethylbenzene or mixtures thereof. More preferably, the solvent is selected from the group consisting of tetrahydrofuran (THF), diethyl ether, heptane and ethylbenzene or mixtures thereof. Most preferably, the solvent is tetrahydrofuran (THF).

For contacting the compound of formula (4) with a second base and a third protective reagent to form a compound of formula (5) the molar ratio of the third protective reagent and compound of formula (4) is typically at least 0.8, preferably at least 0.9, and most preferably at least 1, and generally at most 2, preferably at most 1.8, even more preferably at most 1.6 and most preferably at most 1.5.

A suitable temperature for contacting the compound of formula (4) with a second base and a third protective reagent to form a compound of formula (5) is preferably at least −85° C., more preferably at least −80° C., even more preferably at least −75° C. and most preferably at least −70° C. A suitable temperature at step i. is preferably at most 50° C., more preferably at most 40° C., even more preferably at most 30° C. and most preferably at most 25° C.

Solvents, suitable for contacting the compound of formula (4) with a third oxidizing agent include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably, the solvent is selected from the group consisting of tetrahydrofuran (THF), dichloromethane (DCM) and toluene or mixtures thereof. Most preferably, the solvent is dichloromethane (DCM).

For contacting the compound of formula (4) with a third oxidizing agent the molar ratio of the third oxidizing agent and compound of formula (4) is typically at least 0.8, preferably at least 0.9, and most preferably at least 1, and generally at most 1.5, preferably at most 1.4 and most preferably at most 1.3.

A suitable temperature for contacting the compound of formula (4) with a third oxidizing agent is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

Solvents, suitable for contacting the compound of formula (5) with a third oxidizing agent to obtain a compound of formula (6) include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably the solvent is selected from the group consisting of tetrahydrofuran (THF), dichloromethane (DCM) and toluene or mixtures thereof. Most preferably the solvent is dichloromethane (DCM).

For contacting compound of formula (5) with a third oxidizing agent to obtain a compound of formula (6) the molar ratio of the third oxidizing agent and compound of formula (5) is typically at least 0.8, preferably at least 0.9, and most preferably at least 1, and generally at most 2, preferably at most 1.8, even more preferably at most 1.6, even more preferably at most 1.4 and most preferably at most 1.3.

A suitable temperature for contacting the compound of formula (5) with a third oxidizing agent to obtain a compound of formula (6) is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

In case R⁵ in the compound of formula (6) shall be a protective group, a fourth protective reagent may be added to the compound obtained from contacting a compound of formula (5) with a third oxidizing agent. The molar ratio of the fourth protective reagent and the compound obtained from contacting a compound of formula (5) with a third oxidizing agent is typically at least 0.8, preferably at least 0.9, and most preferably at least 1, and generally at most 2, preferably at most 1.8, even more preferably at most 1.6 and most preferably at most 1.5.

Contacting the compound of formula (6) with a fourth oxidizing agent to obtain the compound of formula (7) may be performed in the presence or absence of a solvent or mixture of solvents.

Solvents, suitable for contacting the compound of formula (6) with a fourth oxidizing agent to obtain the compound of formula (7) include organic solvents or mixtures of organic solvents with water. Preferably, the solvent suitable for contacting the compound of formula (6) with a fourth oxidizing agent to obtain the compound of formula (7) are selected from the group consisting of methanol (MeOH), dimethyl sulfoxide (DMSO), tetrahydrofuran (THF), dichloromethane (DCM), chloroform (CHCl₃), toluene, xylene and benzene or mixtures thereof. More preferably, the solvent is selected from the group consisting of methanol (MeOH) and dimethyl sulfoxide (DMSO) or mixtures thereof. Most preferably, the solvent is methanol (MeOH).

For contacting the compound of formula (6) with a fourth oxidizing agent to obtain the compound of formula (7) the molar ratio of the fourth oxidizing agent and the compound of formula (6) is typically between 1 and 3. Preferably, for contacting the compound of formula (6) with a fourth oxidizing agent to obtain the compound of formula (7) the molar ratio of the fourth oxidizing agent and the compound of formula (6) is preferably at least 1.6, more preferably at least 1.8, most preferably at least 2, and preferably at most 2.8, more preferably at most 2.5 and most preferably at most 2.3.

A suitable temperature for contacting the compound of formula (6) with a fourth oxidizing agent to obtain the compound of formula (7) is preferably at least 0° C., more preferably at least 10° C., even more preferably at least 15° C. and most preferably at least 20° C. A suitable temperature for contacting compound of formula (6) with a fourth oxidizing agent to obtain the compound of formula (7) is preferably at most 60° C., more preferably at most 50° C., even more preferably at most 45° C. and most preferably at most 30° C.

The invention further pertains to a process comprising contacting the compound of formula (4)

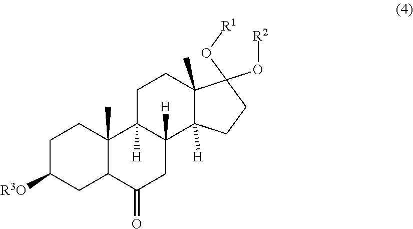

wherein R¹, R² and R³ are protective groups, with a second base and a third oxidizing agent and optionally with a fourth protective reagent to obtain a compound of formula (6), wherein the molar ratio of the third oxidizing agent and the compound of formula (4) is at most 1.5; and
contacting a compound of formula (6)

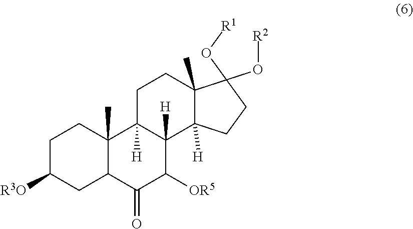

wherein R⁵ is H or a protective group, with a fourth oxidizing agent to obtain the compound of formula (7).
R¹, R², R³, R⁵, the second base, the second protective reagent, the third oxidizing agent, the fourth protective reagent and the fourth oxidizing agent are selected as described above.

Solvents, suitable for contacting the compound of formula (4) with a second base and a third oxidizing agent include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably the solvent is selected from the group consisting of tetrahydrofuran (THF), dichloromethane (DCM) and toluene or mixtures thereof. Most preferably the solvent is dichloromethane (DCM).

For contacting the compound of formula (4) with a second base and a third oxidizing agent the molar ratio of the third oxidizing agent and compound of formula (4) is typically at least 0.8, preferably at least 0.9, and most preferably at least 1, and generally at most 1.5, preferably at most 1.4 and most preferably at most 1.3.

A suitable temperature for contacting the compound of formula (4) with a base and a third oxidizing agent is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

In case R⁵ in compound of formula (6) shall be a protective group, a fourth protective reagent may be added to the compound obtained from either contacting a compound of formula (4) with a second base and a third oxidizing agent or contacting a compound of formula (5) with a third oxidizing agent. The molar ratio of the fourth protective reagent and the compound obtained from either contacting a compound of formula (4) with a second base and a third oxidizing agent or contacting a compound of formula (5) with a third oxidizing agent of formula (6) is typically at least 0.8, preferably at least 0.9, and most preferably at least 1, and generally at most 2, preferably at most 1.8, even more preferably at most 1.6 and most preferably at most 1.5.

The invention further pertains to a process comprising contacting the compound of formula (4)

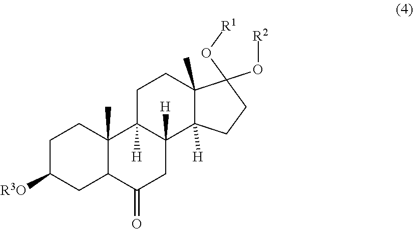

wherein R¹, R² and R³ are protective groups, with a second base and a third oxidizing agent to obtain the compound of formula (7)

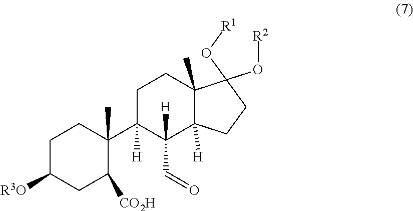

wherein R¹, R² and R³ are protective groups and wherein the molar ratio of the third oxidizing agent and the compound of formula (4) is at least 1.5
R¹, R², R³, the second base, and the third oxidizing agent are selected as described above.

Solvents, suitable for contacting the compound of formula (4) with a second base and a third oxidizing agent to obtain the compound of formula (7) include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably the solvent is selected from the group consisting of tetrahydrofuran (THF), dichloromethane (DCM) and toluene or mixtures thereof. Most preferably the solvent is tetrahydrofuran (THF).

For contacting the compound of formula (4) with a third oxidizing agent to obtain the compound of formula (7) the molar ratio of the third oxidizing agent and compound of formula (4) is typically at least 1.5, preferably at least 1.7, and most preferably at least 2, and generally at most 3, preferably at most 2.8, even more preferably at most 2.6 and most preferably at most 2.5.

A suitable temperature for contacting the compound of formula (4) with a base and a third oxidizing agent to obtain the compound of formula (7) is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

The invention further pertains to a process comprising converting the compound of formula (4) to a compound of formula (7)

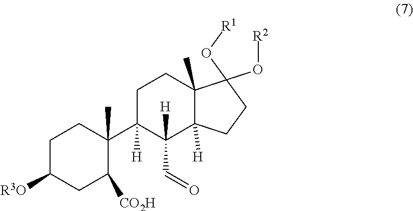

comprising the steps selected from:
i) contacting the compound of formula (4) with a second base and a third protective reagent to form a compound of formula (5)

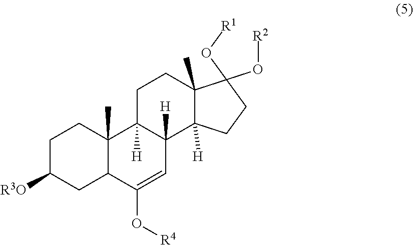

wherein R⁴ is a protective group, and
contacting the compound of formula (5) with a third oxidizing agent and optionally with a fourth protective reagent to obtain a compound of formula (6)

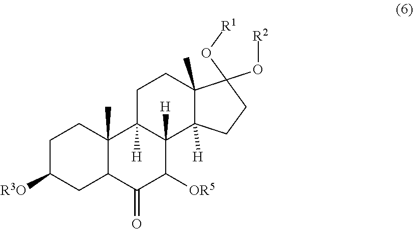

wherein R⁵ is H or a protective group; and
contacting the compound of formula (6) with a fourth oxidizing agent to obtain the compound of formula (7); or

ii) contacting the compound of formula (4) with a second base and a third oxidizing agent and optionally with a fourth protective reagent to obtain a compound of formula (6), wherein the molar ratio of the third oxidizing agent and the compound of formula (4) is at most 1.5; and
contacting a compound of formula (6) with a fourth oxidizing agent to obtain the compound of formula (7); or

iii) contacting the compound of formula (4) with a second base and a third oxidizing agent and optionally with a fourth protective reagent to obtain the compound of formula (7), wherein the molar ratio of the third oxidizing agent and the compound of formula (4) is at least 1.5.

R¹, R², R³,R⁴, R⁵, the second base, the third protective reagent, the third oxidizing agent, the fourth protective reagent and the fourth oxidizing agent are selected as described above.

Contacting the compound of formula (4) with a second base and a third protective reagent to form the compound of formula (5) may be performed in the presence of a solvent or a mixture of solvents. Suitable solvents include anhydrous tetrahydrofuran (THF), 2-methyltetrahydrofuran MeTHF, ether, dioxane, dimethoxyethane (DME), heptane, toluene, xylene or benzene. Preferably the solvent is selected from the group consisting of tetrahydrofuran (THF), diethyl ether, 1,4-dioxane, heptane and ethylbenzene or mixtures thereof. More preferably the solvent is selected from the group consisting of tetrahydrofuran (THF), diethyl ether, heptane and ethylbenzene or mixtures thereof. Most preferably the solvent is tetrahydrofuran (THF).

For contacting the compound of formula (4) with a second base and a third protective reagent to form the compound of formula (5) the molar ratio of the third protective reagent and compound of formula (4) is typically at least 0.8, preferably at least 0.9, and most preferably at least 1, and generally at most 2, preferably at most 1.8, even more preferably at most 1.6 and most preferably at most 1.5.

A suitable temperature for contacting the compound of formula (4) with a second base and a third protective reagent to form a compound of formula (5) is preferably at least −85° C., more preferably at least −80° C., even more preferably at least −75° C. and most preferably at least −70° C. A suitable temperature at step i. is preferably at most 50° C., more preferably at most 40° C., even more preferably at most 30° C. and most preferably at most 25° C.

Solvents, suitable for contacting the compound of formula (4) with a second base and a third oxidizing agent disclosed in step iii. include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably, the solvent is selected from the group consisting of tetrahydrofuran (THF), dichloromethane (DCM) and toluene or mixtures thereof. Most preferably, the solvent is tetrahydrofuran (THF).

Solvents, suitable for contacting the compound of formula (5) with a third oxidizing agent as disclosed in step i. or ii. include organic solvents or mixtures of organic solvents with water. Preferably, suitable solvents are selected from the group consisting of tetrahydrofuran (THF), dichloromethane (DCM), chloroform (CHCl₃), toluene and benzene or mixtures thereof. More preferably the solvent is selected from the group consisting of tetrahydrofuran (THF), dichloromethane (DCM) and toluene or mixtures thereof. Most preferably the solvent is dichloromethane (DCM).

For contacting compound of formula (5) with a third oxidizing agent as disclosed in step i. the molar ratio of the third oxidizing agent and compound of formula (5) is typically at least 0.8, preferably at least 0.9, and most preferably at least 1, and generally at most 2, preferably at most 1.8, even more preferably at most 1.6, even more preferably at most 1.4 and most preferably at most 1.3.

For contacting the compound of formula (4) with a third oxidizing agent as disclosed in step ii. the molar ratio of the third oxidizing agent and compound of formula (4) is typically at least 0.8, preferably at least 0.9, and most preferably at least 1, and generally at most 1.5, preferably at most 1.4 and most preferably at most 1.3.

For contacting the compound of formula (4) with a third oxidizing agent as disclosed in step iii. the molar ratio of the third oxidizing agent and compound of formula (4) is typically at least 1.5, preferably at least 1.7, and most preferably at least 2, and generally at most 3, preferably at most 2.8, even more preferably at most 2.6 and most preferably at most 2.5.

A suitable temperature for contacting the compound of formula (4) with a base and a third oxidizing agent or contacting the compound of formula (5) with a third oxidizing agent is preferably at least −30° C., more preferably at least −20° C., even more preferably at least −10° C. and most preferably at least −5° C., and preferably at most 60° C., more preferably at most 50° C., even more preferably at most 45° C. and most preferably at most 25° C.

In case R⁵ in compound of formula (6) shall be a protective group, a fourth protective reagent may be added to the compound obtained from either contacting a compound of formula (4) with a second base and a third oxidizing agent or contacting a compound of formula (5) with a third oxidizing agent. The molar ratio of the fourth protective reagent and the compound obtained from either contacting a compound of formula (4) with a second base and a third oxidizing agent or contacting a compound of formula (5) with a third oxidizing agent of formula (6) is typically at least 0.8, preferably at least 0.9, and most preferably at least 1, and generally at most 2, preferably at most 1.8, even more preferably at most 1.6 and most preferably at most 1.5.

In steps i. and ii. of the invention a compound of formula (6) is contacted with a fourth oxidizing agent to obtain a compound of formula (7).

The fourth oxidizing agent is a member of the group of compounds which is able to transform the alpha-hydroxy-ketone moiety of compound of formula (6) into two separated functional groups, i.e. an aldehyde and a carboxylic acid, linked to separate cyclic moieties as present in compound of formula (7).

Suitable fourth oxidizing agents of the present invention include percarbonates, perjodates, lead tetraacetate (LTA), osmium complexes, bismuthate, bismuth(III) carboxylates, iodo triacetate, manganic pyrophosphate, manganese dioxide, potassium peroxymonosulfate (KHSO₅), calcium hypochlorite, basic hydrogen peroxide, methylrhenium trioxide, chromium(VI) reagents, ceric ammonium nitrate and vanadium-based heteropolyacids (HPA). Preferably the fourth oxidizing agent of the invention is selected from the group consisting of sodium percarbonate, sodium metaperiodate (NalO₄), lead tetraacetate (LTA), sodium bismuthate (NaBiO₃), bismuth(III)mandelate, iodo triacetate, manganic pyrophosphate, manganese dioxide, potassium peroxymonosulfate (KHSO₅) and calcium hypochlorite, ore preferably from the group consisting of sodium percarbonate, sodium metaperiodate (NalO₄), lead tetraacetate (LTA) and potassium peroxymonosulfate (KHSO₅), even more preferably from the group consisting of sodium metaperiodate and lead tetraacetate (LTA). Most preferably the fourth oxidizing agent of the invention is sodium metaperiodate (NalO₄).

Contacting the compound of formula (6) with a fourth oxidizing agent to obtain the compound of formula (7) may be performed in the presence or absence of a solvent or mixture of solvents.

Solvents, suitable for contacting the compound of formula (6) with a fourth oxidizing agent to obtain the compound of formula (7) include organic solvents or mixtures of organic solvents with water. Preferably, the solvent suitable for contacting compound of formula (6) with a fourth oxidizing agent to obtain a compound of formula (7) are selected from the group consisting of methanol (MeOH), dimethyl sulfoxide (DMSO), tetrahydrofuran (THF), dichloromethane (DCM), chloroform (CHCl₃), toluene, xylene and benzene or mixtures thereof. More preferably the solvent is selected from the group consisting of methanol (MeOH) and dimethyl sulfoxide (DMSO) or mixtures thereof. Most preferably the solvent is methanol (MeOH).

For contacting the compound of formula (6) with a fourth oxidizing agent to obtain the compound of formula (7) the molar ratio of the fourth oxidizing agent and compound of formula (6) is typically between 1 and 3. Preferably, for contacting compound of formula (6) with a fourth oxidizing agent to obtain a compound of formula (7) the molar ratio of the fourth oxidizing agent and compound of formula (6) is preferably at least 1.6, more preferably at least 1.8, most preferably at least 2, and preferably at most 2.8, more preferably at most 2.5 and most preferably at most 2.3.

A suitable temperature for contacting the compound of formula (6) with a fourth oxidizing agent to obtain the compound of formula (7) is preferably at least 0° C., more preferably at least 10° C., even more preferably at least 15° C. and most preferably at least 20° C. A suitable temperature for contacting compound of formula (6) with a fourth oxidizing agent to obtain a compound of formula (7) is preferably at most 60° C., more preferably at most 50° C., even more preferably at most 45° C. and most preferably at most 30° C.

The invention further pertains to a process comprising contacting the compound of formula (7)

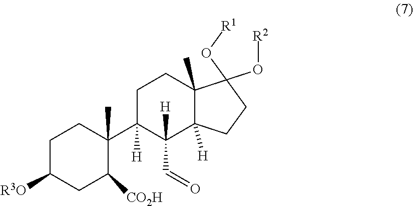

wherein R¹, R² and R³ are protective groups, with a compound of formula (7a)

NH₂—R⁶ (7a)

wherein R⁶ is either H or —OR²², wherein R²² is either H or a protective group, to obtain a compound of formula (8)

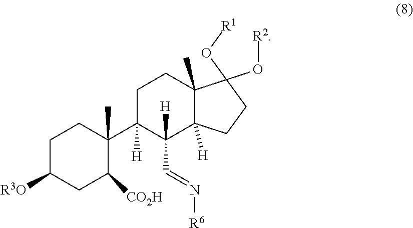

R¹, R², R³ and R⁶ are selected as described above.

The step of contacting the compound of formula (7) with a compound of formula (7a) to obtain the compound of formula (8) may be performed in the presence of a solvent or mixture of solvents. Suitable solvents include water, organic solvents or mixtures of organic solvents with water. Preferably the solvent in the step of contacting the compound of formula (7) with the compound of formula (7a) to obtain the compound of formula (8) is selected from the group consisting of water, methanol, ethanol, 1-butanol, 2-butanol, 1,2-dimethoxy-ethane (DME), tetrahydrofuran (THF) and 1,4-dioxane. More preferably, the solvent is selected from the group consisting of water, methanol, ethanol and tetrahydrofuran (THF) or mixtures thereof. Most preferably, the solvent is tetrahydrofuran (THF).

The molar ratio of the compound of formula (7a) and the compound of formula (7) when contacting the compound of formula (7) with compound of formula (7a) to obtain the compound of formula (8) is typically at least 0.8, preferably at least 0.9, and most preferably at least 1, and generally at most 1.5, preferably at most 1.4 and most preferably at most 1.3.

The temperature at the step of contacting the compound of formula (7) with the compound of formula (7a) to obtain the compound of formula (8) is preferably at least 0° C., more preferably at least 10° C., even more preferably at least 15° C. and most preferably at least 20° C. The temperature at the step of contacting the compound of formula (7) with compound of formula (7a) to obtain a compound of formula (8) is preferably at most 90° C., more preferably at most 70° C., even more preferably at most 50° C. and most preferably at most 35° C.

The invention further pertains to a process comprising contacting the compound of formula (8)

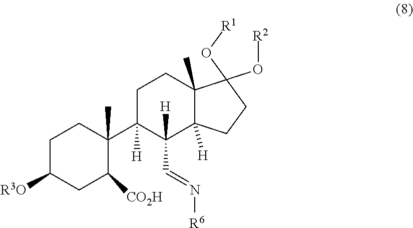

wherein R⁶ is either H or —OR²², wherein R²² is either H or a protective group, with a reducing agent to obtain the compound of formula (9)

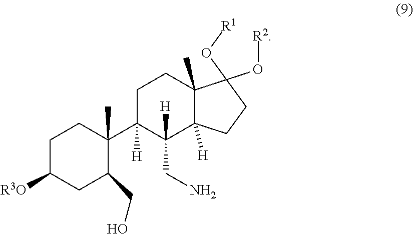

R¹, R², R³ and R⁶ and the reducing agent are selected as described above.

The reducing agent is a member of the group of compounds which is able to transform the imino group of the compound of formula (8) into an amine as present in the compound of formula (8). Preferably, the reducing agent is selected from the group consisting of hydrogen or an ionic or a metallic hydride. More preferably the reducing agent is selected from the group consisting of hydrogen, alane (AlH₃), lithium aluminium hydride (LiAlH₄), sodium hydride (NaH) and diisobutylaluminiumhydride (DIBAL-H), even more preferably selected from the group consisting of hydrogen, alane (AlH₃), lithium aluminium hydride (LiAlH₄)and diisobutylaluminiumhydride (DIBAL-H) and most preferably the reducing agent is alane (AlH₃).

In case hydrogen is selected as reducing agent, catalysts may be present as known in the art. Examples of such catalysts include Ni, Co, Pd, Pt, Ru, Rh, Os, Ir, Os and Fe.

The step of contacting the compound of formula (8) with a reducing agent to obtain the compound of formula (9) may be performed at atmospheric or elevated pressure. Preferably the transformation is performed at atmospheric pressure.

Solvents, suitable for the step of contacting the compound of formula (8) with a reducing agent to obtain the compound of formula (9) include organic solvents or mixtures of organic solvents. Preferably, the solvent for the step of contacting compound of formula (8) with a reducing agent to obtain the compound of formula (9) is selected from the group consisting of 1,2-dimethoxy-ethane (DM E), tetrahydrofuran (THF), chloroform (CHCl₃), toluene and benzene or mixtures thereof. More preferably, the solvent is selected from the group consisting of tetrahydrofuran (THF) and toluene or mixtures thereof. Most preferably, the solvent is tetrahydrofuran (THF).

The invention further pertains to a process comprising the step of contacting a compound of formula (9)

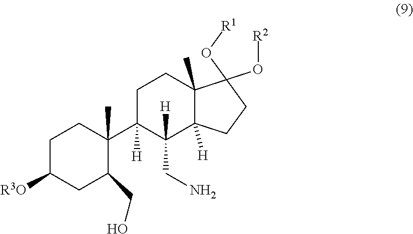

wherein R¹, R² and R³ are protective groups, with a first acid capable of forming a pharmaceutically acceptable salt and optionally a fifth protective reagent to obtain the compound of formula (10)

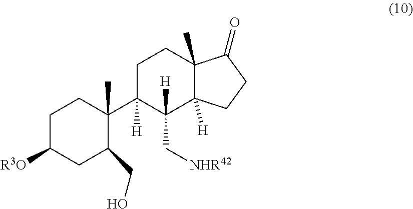

wherein R⁴² is H or a protective group, R⁴³ is H or R³, wherein R³ is a protective group, comprising a counter acid X¹ when R⁴²═H and R⁴³═H.
R¹, R², R³, R⁴² and R⁴³ are selected as described above.

X¹ represents a counter acid which together with the compounds of the present invention, in particular with compound of formula (9) may form a pharmaceutically acceptable salt to obtain compound of formula (10) and which generally may be selected as known in the art. Examples for the selection of X¹ are provided by P. H. Stahl and C. G. Wermuth, editors, Handbook of Pharmaceutical Salts: Properties, Selection and Use, Weinheim/Zürich:Wiley-VCH/VHCA, 2011. Preferably, X¹ is selected from the group consisting of any mineral acid and any organic mono- or di-acid. More preferably, X¹ is selected from the group consisting of hydrochloric acid, hydrobromic acid, sulphuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Even more preferably, X¹ is selected from the group consisting of acetic acid, benzenesulfonic acid, hydrochloric acid, hydrobromic acid, sulphuric acid, phosphoric acid, oxalic acid, tartaric acid and p-toluenesulfonic acid. Even more preferably, X¹ is selected from the group consisting of benzenesulfonic acid and p-toluenesulfonic acid. Most preferably X¹ is p-toluenesulfonic acid.

A first acid represents a counter acid which together with the compounds of the present invention, in particular with the compound of formula (9) may form a pharmaceutically acceptable salt to obtain the compound of formula (10) and which generally may be selected as known in the art. First acid is equivalent to X¹ and selected accordingly.

The fifth protective reagent is a member of the group of compounds which is able to form a protective group for amino groups and is generally selected as known in the art. Examples of suitable fifth protective reagents to obtain protective groups for amino groups are described in T. W. Greene, P. G. M. Wutts “Protective Groups in Organic Synthesis”, John Wiley & Sons, 5^{th} Ed., 2014, pages 895-1115. Preferably, the fifth protective reagent is selected from the group consisting of methyl chloroformate, fluorenylmethyloxycarbonyl chloride (Fmoc-Cl), 1,1-dioxobenzo[b]thiophen-2-ylmethyl chloroformate, 2-(trimethylsilyl)ethyl chloroformate, 2-phenylethyl chloroformate, di-tert-butyldicarbonat, 1-adamantyl chloroformate (1-Adoc-Cl), 2-adamantyl chloroformate (2-Adoc-Cl), benzyl bromide, acetic anhydride, acetyl chloride, benzoylchloride, trifluoroacetic anhydride, ethyl formate and phenyl formate. More preferably, the fifth protective reagent is selected from the group consisting of di-tert-butyldicarbonat, 1-adamantyl chloroformate (1-Adoc-Cl), 2-adamantyl chloroformate (2-Adoc-Cl), benzyl bromide, acetic anhydride, acetyl chloride, benzoylchloride, trifluoroacetic anhydride, ethyl formate and phenyl form ate. Even more preferably, the fifth protective reagent is selected from the group consisting of di-tert-butyldicarbonat, 1-adamantyl chloroformate (1-Adoc-Cl), benzyl bromide, acetic anhydride, trifluoroacetic anhydride and ethyl formate. Most preferably, the fifth protective reagent is di-tert-butyldicarbonat.

Solvents, suitable for contacting the compound obtained from contacting a compound of formula (9) with a first acid capable of forming a pharmaceutically acceptable salt with a fifth protective reagent include organic solvents or mixtures of organic solvents. Preferably, the solvent is selected from the group consisting of methanol, ethanol, acetonitrile, acetone, tetrahydrofuran (THF) and dichloromethane (DCM) or mixtures thereof. More preferably, the solvent is selected from the group consisting of tetrahydrofuran (THF) and dichloromethane (DCM) or mixtures thereof. Most preferably, the solvent is dichloromethane (DCM).

The molar ratio of the fifth protective group and the compound of formula (9) when contacting the compound obtained from contacting the compound of formula (9) with a first acid capable of forming a pharmaceutically acceptable salt with the fifth protective reagent is typically at least 0.8, preferably at least 0.9, and most preferably at least 1, and generally at most 1.5, preferably at most 1.4 and most preferably at most 1.3.

Generally, the temperature at the step of contacting the compound obtained from contacting the compound of formula (9) with a first acid capable of forming a pharmaceutically acceptable salt with a fifth protective reagent ranges from −10° C. up to reflux of the solvent or solvent mixture. The temperature at the step of contacting the compound obtained from contacting the compound of formula (9) with a first acid capable of forming a pharmaceutically acceptable salt with a fifth protective reagent is preferably at least 0° C., more preferably at least 10° C., even more preferably at least 15° C. and most preferably at least 20° C. The temperature at the step of contacting the compound of formula (7) with the compound of formula (7a) to obtain the compound of formula (8) is preferably at most 90° C., more preferably at most 70° C., even more preferably at most 50° C. and most preferably at most 35° C.

The invention further pertains to a process for preparing a compound of formula (12)

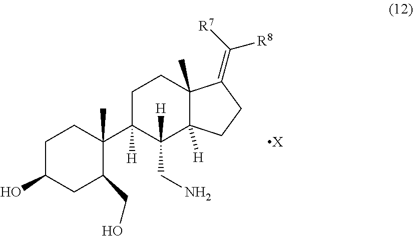

wherein R⁷ and R⁸ are each independently selected from H, halogen, alkyl, cycloalkyl, alkoxy, aryloxy, aryl, or alkylaryl and X is a counter acid, comprising the conversion of a compound of formula (11)

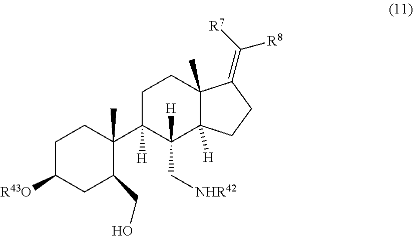

wherein R⁷ and R⁸ are each independently selected from H, halogen, alkyl, aryl, or arylalkyl, R⁴² is H or a protective group, R⁴³ is H or R³, wherein R³ is a protective group, with a second acid capable of forming a pharmaceutically acceptable salt to obtain the compound of formula (12).
R³, R⁷, R⁸ and second acid are selected as described above.

The invention further pertains to a process for preparing a compound of formula (12)

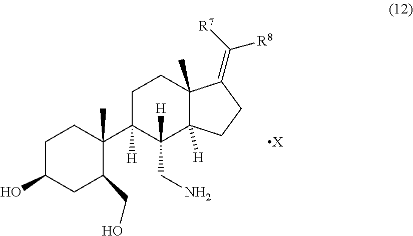

wherein R⁷ and R⁸ are each independently selected from H, halogen, alkyl, cycloalkyl, alkoxy, aryloxy, aryl, or arylalkyl and X is a counter acid,
comprising the steps of
a. contacting dehydroepiandrosteron (DHEA) with a first protective reagent to form a ketal of compound of formula (1)

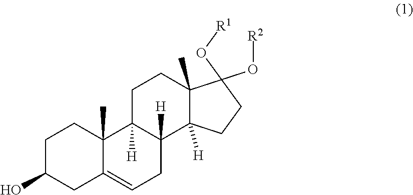

wherein R¹ and R² independently are protective groups or together form one protective group;
b. contacting a compound of formula (1) with a second protective reagent to form a compound of formula (2)

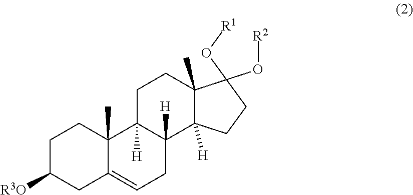

wherein R¹ and R² are as defined for compound of formula (1) and wherein R³ is a protective group;
c. contacting a compound of formula (2) with a borane and a first oxidizing agent to obtain a compound of formula (3)

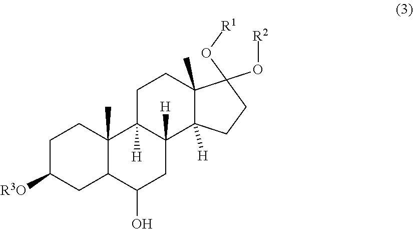

wherein R¹ and R² are as defined for compound of formula (1) and R³ is as defined for compound of formula (2);
d. contacting a compound of formula (3) with a second oxidizing agent to obtain a compound of formula (4)

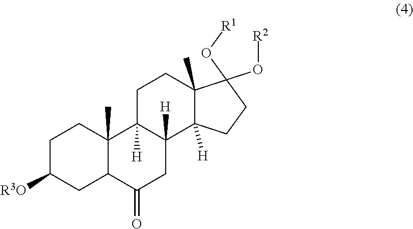

wherein R¹ and R² are as defined for compound of formula (1) and R³ is as defined for compound of formula (2);
e. converting the compound of formula (4) to a compound of formula (7)

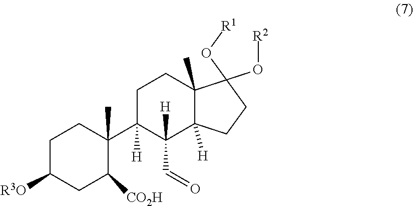

wherein R¹, R² and R³ are protective groups, comprising the steps selected from:
   i) contacting the compound of formula (4) with a second base and a third protective reagent to form a compound of formula (5)

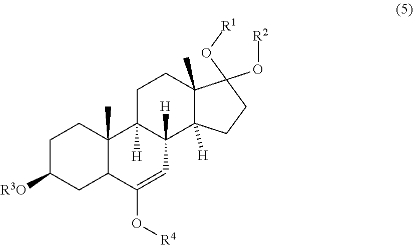

wherein R⁴ is a protective group, and
contacting the compound of formula (5) with a third oxidizing agent and optionally with a fourth protective reagent to obtain a compound of formula (6)

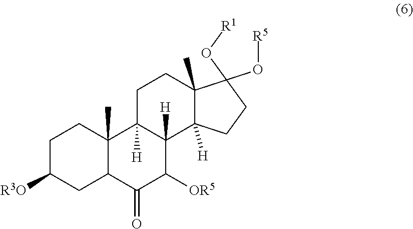

wherein R⁵ is H or a protective group; and
contacting the compound of formula (6) with a fourth oxidizing agent to obtain the compound of formula (7); or
   ii) contacting the compound of formula (4) with a second base and a third oxidizing agent and optionally with a fourth protective reagent to obtain a compound of formula (6), wherein the molar ratio of the third oxidizing agent and the compound of formula (4) is at most 1.5; and contacting a compound of formula (6) with a fourth oxidizing agent to obtain the compound of formula (7); or
   iii) contacting the compound of formula (4) with a second base and a third oxidizing agent and optionally with a fourth protective reagent to obtain the compound of formula (7), wherein the molar ratio of the third oxidizing agent and the compound of formula (4) is at least 1.5;
f. contacting a compound of formula (7) with a compound of formula (7a)

NH₂—R⁶ (7a)
wherein R⁶ is either H or —OR^{6a}, wherein R^{6a} is either H or a protective group, to obtain a compound of formula (8)

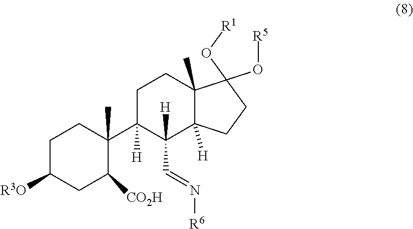

wherein R¹ and R² are as defined for compound of formula (1), R³ is as defined for compound of formula (2) and R⁶ is as defined above;
g. contacting a compound of formula (8) with a reducing agent to obtain a compound of formula (9)

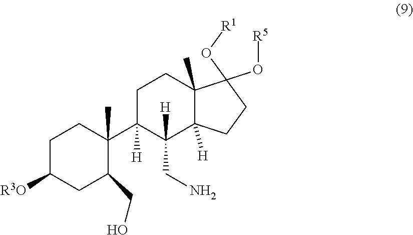

wherein R¹ and R² are as defined for compound of formula (1), R³ is as defined for compound of formula (2);
h. contacting a compound of formula (9) with a first acid which forms a non-toxic salt and optionally a fifth protective reagent to obtain a compound of formula (10)

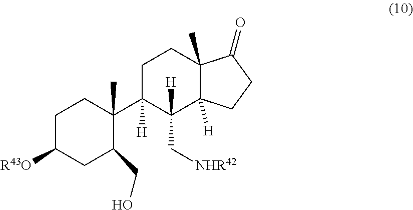

wherein R⁴² is H or a protective group, R⁴³ is H or R³, wherein R³ is a protective group, comprising a counter acid X¹ when R⁴²═H and R⁴³═H;
i. contacting a compound of formula (10) with an olefination reagent to obtain a compound of formula (11)

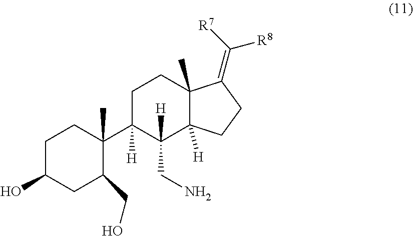

wherein R⁷ and R⁸ are each independently selected from H, halogen, alkyl, aryl, or arylalkyl;
j. further conversion of a compound of formula (11) with a second acid which forms a non-toxic salt to obtain a compound of formula (12).
   R¹, R², R³, R⁴, R⁵, R⁶, R⁷, R⁸, R⁴², R⁴³, borane, first oxidizing agen, second oxidizing agent, third oxidizing agent, fourth oxidizing agent, first base, second base, third protective reagent, fourth protective reagent, fifth protective reagent, reducing agent, olefination reagent, first acid, and second acid are selected as described above.

In step a. dehydroepiandrosteron (DHEA) is contacted with a first protective reagent optionally in the presence of a catalyst to form a ketal.

With the term “ketal” of is meant a functional group with the following general connectivity R₂C(OR′)₂, where both R′ groups are organic fragments. The central carbon atom has four bonds to it, and is therefore saturated and has tetrahedral geometry. The two R′O groups, in the present invention R¹O and R²O may be equivalent to each other or not. The two R groups can be equivalent to each other (a “symmetric ketal”) or not (a “mixed ketal”) and may form acyclic or cyclic ketals. Ketalization is typically acid catalysed with elimination of water. As a reaction to create a ketal proceeds, water typically must be removed from the reaction mixture, for example, with a Dean-Stark apparatus. The formation of ketals reduces the total number of molecules present and therefore is not favourable with regards to entropy. A way to improve this may be to use an orthoester, e.g. trimethyl orthoformate, trimethylorthoacetate or triethylorthoacetate as a source of alcohol. The reaction equilibrium may be shifted to the ketal when water is removed from the reaction system. Examples of such water removal include azeotropic distillation and trapping water with molecular sieves or aluminium oxide.

With the term “ketalization” is meant any reaction that yields a ketal.

The catalyst to obtain a ketal can be any suitable catalyst known in the art. Commonly used catalysts for ketalization are acids. Preferably the catalyst to obtain a ketal in step a. of the present invention is selected from the group consisting of protic inorganic acids, inorganic metal salts or complexes, organic protic acids, acidic ionic liquids or molecular sieves. Preferably, the catalyst to obtain a ketal in step a. of the present invention is selected from the group consisting of iodine, HCl, FeCl₃, TiCl₄, Amberlyst-15, HZSM-5, MCM-41, montmorillonite, Bi(NO₃)₃, bismuth(III) subnitrate (BSN), BiCl₃, BiBr₃, 2, 3-dichlor-5,6-dicyano-1,4-benzochinon (DDQ), N-bromosuccinimide (NBS), Sc(NTf₂)_{3,} p-toluenesulfonic acid, tetrabutylammonium tribromide (TBATB), perchloric acid or camphor-12 sulfonic acid. More preferably, the catalyst in step a. of the present invention is selected from the group consisting of p-toluenesulfonic acid or camphor-12 sulfonic acid. Most preferably, the catalyst is camphor-12 sulfonic acid.

In the process of the invention, the first protective reagent may react to form the protective groups of R¹ and R². The first protective reagent may be one reagent or a combination of two or more reagents. Preferably, the first protective reagent is an orthoester or an alcohol. More preferably the first protective reagent to form a ketal is a monohydric or a polyhydric alcohol. Even more preferably, the first protective reagent to form a ketal is selected from the group consisting of methanol, ethanol, isopropyl alcohol, butyl alcohol, cyclohexanol, phenol, 1,2-ethanediol or 1,3-propanediol. Even more preferably, the first protective reagent to from a ketal is 1,3-propanediol or 1,2-ethanediol. Most preferably, the first protective reagent to from a ketal is 1,2-ethanediol.

“Polyhydric alcohols” have more than one hydroxyl (—OH) groups. The simplest example of an alcohol with more than one hydroxyl group is methanediol or methylene glycol, HOCH₂OH.

An alcohol that has one hydroxyl group is called “monohydric”. Monohydric alcohols include methanol, ethanol, and isopropanol.

Ketalization of dehydroepiandrosteron (DHEA) to give the compound of formula (1), step a. of the invention, can be performed in the absence or presence of a solvent or mixtures of solvents. Suitable solvents for ketalization are generally known in the art. Preferred solvents are selected from the group consisting of benzene, toluene or xylene. The most preferred solvent is toluene.

Reaction temperature of the ketalization reaction of dehydroepiandrosteron (DHEA) to give the compound of formula (1) is not critical. Generally, the reaction temperature ranges from 0° C. up to the reflux temperature of the solvent or solvent mixture. Preferably, the reaction temperature is at least 5° C., more preferably at least 10° C., and most preferably at least 20° C. Preferably, the reaction temperature is at most 115° C., more preferably the reaction temperature is at most 80° C., even more preferably at most 60° C., even more preferably at most 45° C., and most preferably at most 35° C.

The compound of formula (1) may be purified. For example, the compound of formula (1) may be purified by trituration. With “trituration” is meant a process used to purify crude chemical compounds containing soluble impurities. Therefore, a solvent may be chosen in which the desired product is insoluble and the undesired by-products are very soluble. The crude material may be washed with the solvent and filtered, leaving the purified product in solid form and any impurities in solution.

A solvent for purification of the compound of formula (1) resulting from step a. may be selected from non-polar solvents. More preferably a solvent for purification of the crude compound of formula (1) is selected from the group consisting of pentane, cyclopentane, hexane, or cyclohexane. Most preferably, the solvent for purification of the compound of formula (1) is hexane.

In step b. of the process of the invention, the compound of formula (1) is contacted with a second protective reagent, optionally in the presence of a first base, to form the compound of formula (2).

The second protective reagent suitable to obtain the protective group of R³ is generally known in the art. Examples of second protective reagents include the ones described in T. W. Greene, P. G. M. Wutts “Protective Groups in Organic Synthesis”, John Wiley & Sons, 5^{th} Ed., 2014, pages 17-374.

Preferably, the second protective reagent is selected from the group consisting of alkylsulfate (CH₃SO₄), alkyliodide (CH₃l), diazoalkane (CH₂N₂), dialkylhydrogenphosphite ((MeO)₂POH), isobutylene (C₄H₈), allylbromide, trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl), tri-iso-propylsilyloxymethylchloride, chloromethylmethylether (CMME), bromomethylmethylether, iodomethylmethylether, dimethoxymethane (CH₂(OMe)₂), diethoxyethane (CH₂(OEt)₂ (Phenyldimethylsilyl)methoxymethylchloride (SMOMCl), ethylvinylether, 2-(chloromethoxy)-2-methylpropane (t-BuOCH₂Cl), ((tert-butoxymethyl)sulfonyl)benzene (t-BuOCH₂SO₂Ph), 4-pentenyloxymethylchloride (POMCl), 2-methoxyethoxymethyl chloride (MEMCl), 2,2, 2-trichloroethoxymethoxychloride, 2,2,2-trichloroethoxymethoxybromide, (4-methoxyphenoxy)methylchloride (p-AOMCl), p-methoxybenzyloxymethylchloride (PMBMCl), benzyloxymethylchloride (PhCH₂OCH₂OCl), p-methoxyphenol, p-methoxybenzyl 2,2, 2-trichloroacetimidate, guaiacolmethylchloride (2-MeOC₆H₄OCH₂Cl), acetic anhydride (Ac₂O), benzoylchloride (BzCl), benzylbromide, benzylchloride, iodomethylmethylsulfane (CH₃SHC₂1), chloromethylmethylsulfane (CH₃SHO₂Cl), dimethylsulfide (CH₃SCH₃), oxalylchloride (0001)₂, pivaloyl chloride (PivCl), 3,4-dihydropyran or tritylchloride. More preferably the second protective agent is selected from the group consisting of trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl), or tri-iso-propylsilyloxymethylchloride. Even more preferably the second protective agent is selected from the group consisting of trimethylchlorosilane (TMSCl), trimethylsilyltriflate (TMSOTf), trimethylsilylcyanide (TMSCN), trimethylsilyldiethylamine, triethylsilylchloride (TESCl), triisopropylsilylchloride (TIPSCl), t-butyldimethylsilylchloride (TBDMSCl), t-butyldiphenylsilylchloride (TBDPSCl). Most preferably, the second protective agent is t-butyldimethylsilylchloride (TBDMSCl).

In step b. of the process of the invention, a first base may be present. Preferably, the first base is selected from the group consisting of imidazole, 4-dimethylaminopyridine (DMAP), pyridine, 2,6-Lutidine, 4-(1-pyrrolidinyl)pyridine (PPY), pyrrole, 1, 4-diazabicyclo[2.2.2]octane (DABCO), trimethylamine and N,N-diisopropylethylamine (DIPEA) or mixtures thereof. More preferably, the first base is selected from the group consisting of imidazole, 4-dimethylaminopyridine (DMAP) and 4-(1-pyrrolidinyl)pyridine (PPY) or mixtures thereof. Even more preferably, the first base is selected from the group consisting of imidazole and 4-dimethylamino-pyridine (DMAP). Even more preferably, the first base is imidazole in the presence dimethylamino-pyridine (DMAP). Most preferably, the first base is imidazole.

Step b. of the present invention may be performed in the presence of a solvent or a mixture of solvents. Solvents, suitable in step b. include aprotic organic solvents. Such suitable solvents include toluene, benzene, xylene, tetrahydrofuran (THF), dichloromethane (DCM), dimethylformamide (DMF), pyridine or acetonitrile (MeCN). Preferably, the solvent in step b. of the present invention is selected from the group consisting of toluene, benzene, xylene, dichloromethane (DCM), dimethylformamide (DMF), pyridine or acetonitrile (MeCN). More preferably, the solvent in step b. of the present invention is selected from the group consisting of dichlormethane (DCM) or dimethylformamide (DMF). Most preferably, the solvent is dimethylformamide (DMF).

One reliable and rapid procedure is the protocol, described by E. J. Corey (J. Am. Chem. Soc. 1972, 94, 6192) in which the alcohol is reacted with a silyl chloride and imidazole at high concentration in DMF.

In step b. the molar ratio of the second protective reagent and compound of formula (1) is typically at least 0.8, preferably at least 0.9, and most preferably at least 1, and generally at most 2, preferably at most 1.8, even more preferably at most 1.6 and most preferably at most 1.5.

Generally, the reaction temperature in step b. ranges from about −78° C. up to the reflux temperature of the solvent. Preferably the reaction temperature in step b. is above −10° C. More preferably, the reaction temperature in step b. is above 0° C. Even more preferably the reaction temperature in step b. is above 10° C. Most preferably the reaction temperature in step b. is above 20° C. Preferably, the reaction temperature in step b. is below 115° C. More preferably the reaction temperature in step b. is below 80° C. Even more preferably, the reaction temperature in step b. is below 60° C. Even more preferably, the reaction temperature in step b. is below 45° C. Most preferably, the reaction temperature in step b. is below 35° C.

In step c. the compound of formula (2) is contacted with a borane and a first oxidizing agent to obtain the compound of formula (3).

With the term “borane” is meant a member of the group of compounds with the generic formula of BₓH_{y} as well as substituted equivalents thereto which are able to act as hydroborating reagents.

Preferably, the borane in step c. of the invention is selected from the group consisting of diborane (B₂H₆), monosubstituted boranes or disubstituted boranes. More preferably, the borane is selected from the group consisting of diborane (B₂H₆), monochloroborane, monobromoborane, monoiodoborane, monophenylborane, thexylboran (ThBH₂), monoisopinocamphenylboran (IpcBH₂), diisopinocampheylboran (Ipc)₂BH, 9-borabicyclo[3.3.1]nonane (9-BBN), bis-3-methyl-2-butylborane (disiamylborane; Sia₂BH), dimethoxyborane, dicyclohexylborane (Chx₂BH), diphenylborane, dimesitylborane (C₆H₂Me₃)₂BH or catecholboran. Most preferably, the borane is diborane (B₂H₆).

Step c. of the present invention may be performed in the presence of a solvent or a mixture of solvents. Solvents for the hydroboration reaction in step c for example include ethers, toluene, benzene and xylene. Preferably, the solvent in the hydroboration reaction in step c. of the present invention is selected from ethers. Preferably, the solvent in the hydroboration reaction in step c. of the present invention includes dimethyl ether, diethyl ether, tetrahydrofuran, methyl tert.-butyl ether, monoglyme, diglyme (as used herein, glyme is defined as a polyethylene glycol dimethyl ether having the general formula CH₃(OC₂H₄)ₙOCH₃ where n is an integer from 1 to 4), and mixtures thereof. Preferably, the solvent is selected from the group consisting of diethyl ether, tetrahydrofuran or methyl tert.-butyl ether. More preferably, the solvent is selected from the group consisting of tetrahydrofuran or methyl tert.-butyl ether. Most preferably, the solvent is tetrahydrofuran.

The amount of solvent used is not critical; however, sufficient amounts of solvent should be used to dissolve the reactants and prevent excessive heat build-up during the reaction.

In carrying out the hydroboration reaction it is preferred to use a substantially stoichiometric amount of reactants. Use of an excess of either reactant is not detrimental but causes unnecessary recycle of unused reactant.

Reaction temperature of the hydroboration reaction is not critical. Generally, the reaction temperature in step c. of the present invention ranges from about −78 C. up to the reflux temperature of the solvent. Preferably, the reaction temperature in step c. is at least −10° C., more preferably at least 0° C., even more preferably at least 10° C. and most preferably at least 20° C. Preferably, the reaction temperature in step c. is at most 115° C., more preferably at most 80° C., even more preferably at most 60° C., even more preferably at most 45° C. and most preferably at most 35° C.

Use of the hydroboration reaction in step c. of the invention is made to transform a double bond in an unsaturated cyclic moiety of the compound of formula (2) to an organoborane which may further be transformed to an alcohol with an oxidizing agent (i.e. first oxidizing agent).

The first oxidizing agent is a member of the group of compounds which is able to oxidize the organoborane moiety of the compound of formula (2) which may be obtained by hydroboration reaction and thus may form a C-O group, e.g an alcohol. The first oxidizing agent of the present invention includes sodium perborate (NaBO₃) and hydrogen peroxide (H₂O₂). More preferably, the first oxidizing agent is selected from the group consisting of sodium perborate (NaBO₃) and hydrogen peroxide (H₂O₂). Most preferred, as first oxidizing agent of the invention is sodium perborate (NaBO₃).

Solvents, suitable for contacting the organoborane moiety of compound of formula (2) with the first oxidizing agent, are selected as described for the solvents suitable for the hydroboration reaction above. Preferably, the solvent for the hydroboration reaction and contacting the organoborane moiety of compound of formula (2) with the first oxidizing agent is the same.

Generally, the reaction temperature for contacting the organoborane moiety of compound of formula (2) with the first oxidizing agent ranges from −20° C. up to reflux of the solvent or solvent mixture. Preferably, the reaction temperature is at least −10° C., more preferably at least 0° C., even more preferably at least 10° C. and most preferably at least 20° C. Preferably, the reaction temperature is at most 70° C., more preferably at most 60° C., even more preferably at most 45° C. and most preferably at most 30° C.

In step of contacting the organoborane moiety of compound of formula (2) with the first oxidizing agent, the molar ratio of the first oxidizing agent and the organoborane moiety of the compound of formula (2) is typically at least 0.8, preferably at least 0.9, and most preferably at least 1, and generally at most 2, preferably at most 1.8, even more preferably at most 1.6 and most preferably at most 1.5.

The invention further pertains to a process wherein step a. and step b. may be performed without intermediate isolation of the compound of formula (1).

The invention further pertains to a process wherein step c. and step d. may be performed without intermediate isolation of the compound of formula (3).

The invention further pertains to a process wherein step e. may be performed without intermediate isolation of the compounds of formula (5) and formula (6).
The invention further pertains to a process wherein step f., step g. and step h. may be performed without intermediate isolation of the compounds of formula (8) and formula (9).

The invention further pertains to a process wherein each or any of step a. and step b., step c. and step d., step e., and step f. and step g. and step h. may be performed without isolation of intermediates formed.

The invention further pertains to a compound of formula (13):

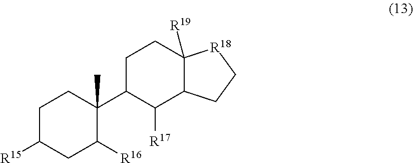

wherein:
R¹⁵ is —R²⁰—OR²¹;
R¹⁶ is —R²⁰—OR²¹, —R²⁴—C(O)OR²⁵;
R¹⁷ is —CH₂O, or —CH₂NHR⁶; or
R¹⁶ and R¹⁷ together form —R²⁶—C(O)—CH₂—, —R²⁷—COR²⁸═CH—, —R²⁹—C(O)—CHOH—,
—R³⁰C(NR³¹)—CHR³²—, —R³³—C(O)—Z—CH R³⁴—;
R¹⁸ is —C(O)—, —C(R³⁶R³⁷)—, wherein R³⁶ is OR⁴⁰ and R³⁷ is OR⁴¹;
—C(CR³⁸R³⁹)—, wherein each R³⁸ and R³⁹ independently are C1-C8 alkyl, aryl, or alkylaryl;
R¹⁹ is C1-C4 alkyl;
each R²⁰, R²⁴, R²⁶, R²⁷, R²⁹, R³⁰, R³³ is independently a direct bond or a methylene;
each R²¹, R²⁵ is independently hydrogen or C1-C8 alkyl;
each R⁶, R³¹, R³², R³⁴ is either H or —OR²², wherein R²² is either H or a protective group; R²⁸ is a protective group;
each R⁴⁰ and R⁴¹ is independently H or a protective group;
or R⁴⁰ and R⁴¹ together form a protective group;
Z is O or NR³⁵, wherein R³⁵ is either H or a protective group;
or a stereoisomer, enantiomer or tautomer thereof or a mixture thereof;
or a pharmaceutically acceptable salt thereof.

Preferably, R³⁸ and R³⁹ are independently C1-C4 alkyl, more preferably methyl. Protective groups R²², R²⁸, R⁴⁰, R⁴¹, are selected as described for R³ above. Protective group R³⁵ is selected as described for R⁴² above.

Suitable intermediates in alternative processes to obtain a compound of formula (12) include

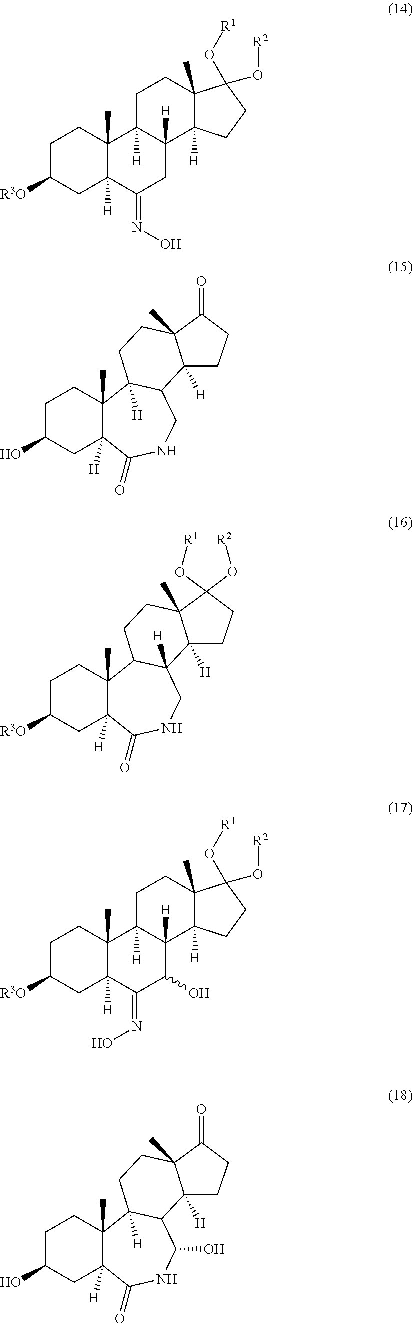

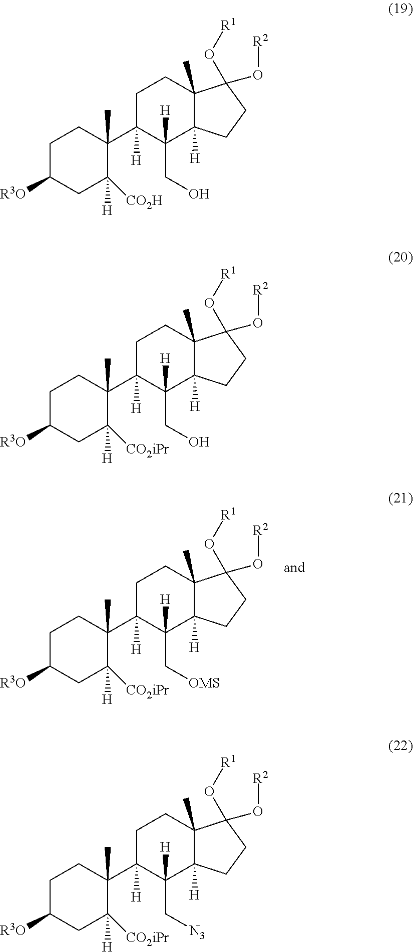

wherein R¹, R² and R³ are protective groups, iPr is isopropyl and Ms is mesyl.

Each of these intermediates (compounds of formula 14-22) can be produced starting from the compound of formula (4) and each of these intermediates can be transformed to the compound of formula (9).

The invention further pertains to a compound of formula (4)

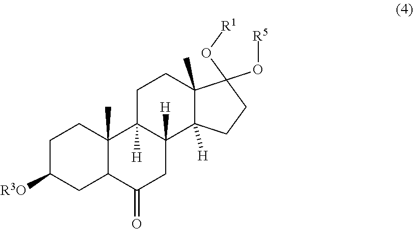

wherein R¹, R² and R³ are protective groups.

The invention further pertains to a compound of formula (5)

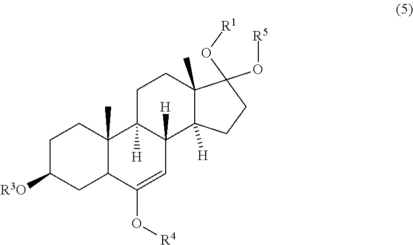

wherein R¹, R², R³ and R⁴ are protective groups.

The invention further pertains to a compound of formula (6)

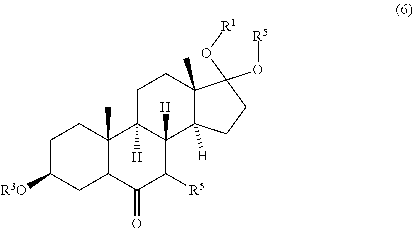

wherein R¹, R² and R³ are protective groups, and R⁵ is H or a protective group.

The invention further pertains to a compound of formula (7)

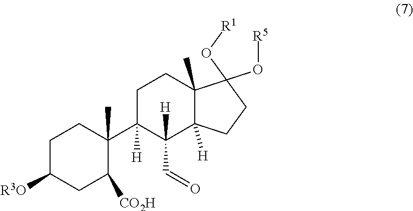

wherein R¹, R² and R³ are protective groups.

The invention further pertains to a compound of formula (8)

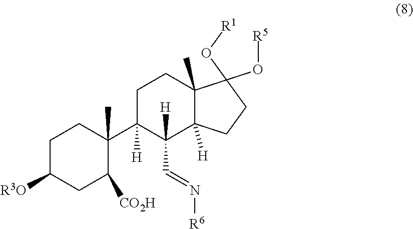

wherein R¹, R² and R³ are protective groups and R⁶ is either H or —OR²², wherein R²² is either H or a protective group.

The invention further pertains to a compound of formula (9)

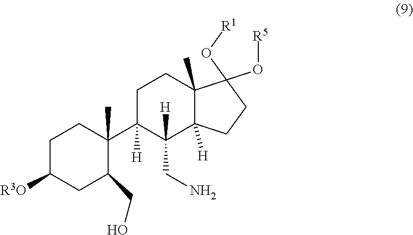

wherein R¹, R² and R³ are protective groups.

The invention further pertains to a compound of formula (10)

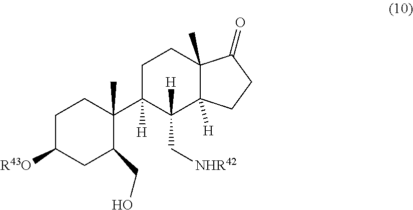

wherein R⁴² is H or a protective group, R⁴³ is H or R³, wherein R³ is a protective group, comprising a counter acid X¹ when R⁴²═H and R⁴³═H.

The invention further pertains to any one of compounds 4-9, 14, 16, 17, and 19-22 wherein R¹ and R² together form —CH₂—CH₂—.

The inventive compounds may exist as single stereoisomers, racemates and/or mixtures of enantiomers and/or diastereomers. All such single stereoisomers, racemates and mixtures thereof are intended to be within the scope of the present invention. In a preferred aspect, the inventive compounds are used in optically pure form.

All intermediates appearing in the process of the invention may be isolated as solids. In particular compounds of formulae (4), (5), (6), (7), (8), (9) and (10) may be obtained and isolated as solids. The atoms, molecules or ions which make up solids may be arranged in an orderly repeating pattern, or irregularly. Materials whose constituents are arranged in a regular pattern are known as crystals. In other materials, there is no long-range order in the position of the atoms. These solids are known as amorphous solids.

The present invention is illustrated by, but not intended to be limited to, the following examples.

### EXAMPLE 1

### Preparation of (3S,8R,9S,10R,13S,14S)-10,13-dimethyl-1,2,3,4,7,8,9,10,11,12,13,14,15,16-tetradecahydrospiro[cyclopenta[a]phenanthrene-17,2′-[1,3]dioxolan]-3-ol (compound of formula (1′), step a.)

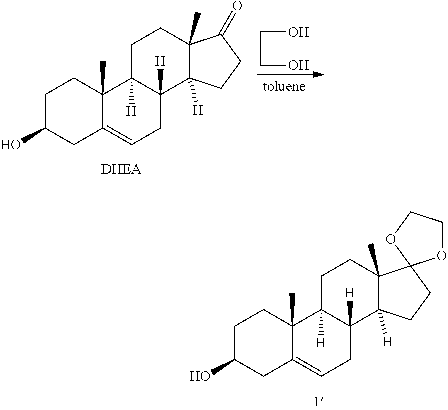

A reactor was charged with 100 g (1.0 eq.) dehydroepiandrosteron (DHEA), 800.0 ml (8.0 vol) toluene, 43 g (3.6 eq.) ethylene glycol and 500 mg camphor-12-sulfonic acid. The reaction mixture was heated to approximately 111° C. and ethylene glycol was removed. Half of the volume of the reaction mixture was separated by distillation and allowed to cool to ambient temperature. To this solution 400 ml (4.0 vol) of hexane were charged and mixed for 2 hours. The solids were isolated by filtration, washed four times each with 100 ml (1.0 vol) of hexane and dried under vacuum at 50° C. to afford 119 g of compound of formula (1′) with 100% yield.

### EXAMPLE 2

### Preparation of tert-butyl (((3S,8R,9S,10R,13S,14S)-10,13-dimethyl-1,2,3,4,7,8,9,10,11,12,13,14,15,16-tetradecahydrospiro[cyclopenta[a]phenanthrene-17,2′-1,3dioxolan]-3-yl)oxy)dimethylsilane (compound of formula (2′), step b

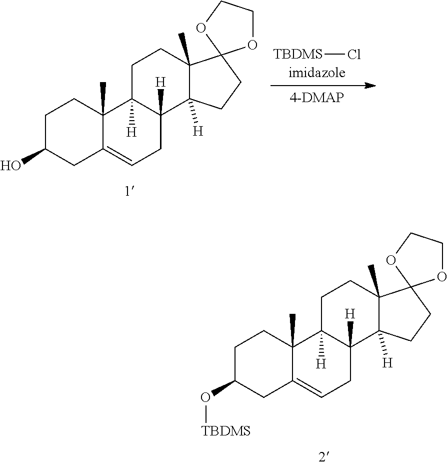

A reactor was charged with 100.7 g (1.0 eq.) compound of formula (1′), 24.74 g (1.4 eq.) imidazole, 11.1 g (0.3 eq.) 4-(dimethylamino)-pyridin (4-DMAP) and 504 ml (5.0 vol) dichloromethane (DCM). The reaction mixture was mixed until a complete solution was obtained. Subsequently the reaction mixture was cooled to −10° C. and 57 g (1.25 eq.) tert-butyldimethylsilyl chloride (TBDMS-Cl) slowly charged while maintaining the temperature below 15° C. The reaction contents were then allowed to warm to ambient temperature. Thereafter, the reaction mixture was diluted with 1,510 ml (15 vol) methyl-tert-butylether (MTBE) and extracted with 805.6 ml (8.0 vol) H₂O. After phase separation the organic phase was washed with a solution of 503.5 ml (5.0 vol) H₂O and 15.91 g (0.25 eq.) citric acid monohydrate, 201.4 ml (2.0 vol) aqueous solution of sodium bicarbonate (NaHCO₃ aq. solution), 201.4 ml (2.0 vol) aqueous solution of sodium chloride (brine), dried over sodium sulphate, separated by filtration, rinsed with methyl-tert-butylether (MTBE) and concentrated to dryness under vaccum at 45° C. to afford 137.8 g of compound of formula (2′) with 100% yield.

### EXAMPLE 3

### Preparation of (3S,5S,8R,9S,10R,13S,14S)-3-((tert-butyldimethylsilyl)oxy)-10,13-dimethylhexadecahydrospiro[cyclopenta[a]phenanthrene-17,2′-[1,3]dioxolan]-6-ol (compound of formula (3′), step c.)

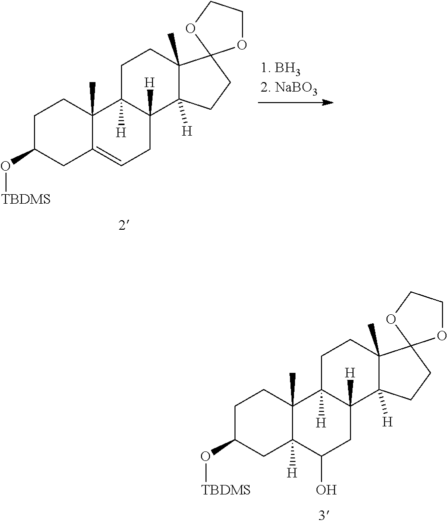

A reactor was charged with a solution of 136.6 g (1.0 eq.) compound of formula (2′) in 683 ml (5.0 vol) tetrahydrofuran (THF) anhydrous. The solution was cooled to −30° C., slowly charged with 336 ml (1.1 eq.) borane (BH₃) in tetrahydrofuran (THF) and mixed at ambient temperature until complete conversion, monitored by chromatography, was achieved. Thereafter, the reaction mixture was quenched by drop wise addition of 1,319 ml (9.7 vol) H₂O and 183.10 g (6.0 eq.) of sodium perborate (NaBO₃) and mixed at ambient temperature until complete conversion to compound of formula (3′) was achieved, which took approximately 10 hours. Subsequently 2,732 ml (10 vol) methyl-tert-butylether (MTBE) and 1,366 ml (10 vol) H₂O were charged to the reactor and mixed. Any remaining excess solids where removed from the present bi-phasic mixture by filtration and the remaining bi-phasic mixture was rinsed with 800 ml (5.9 vol) H₂O. Subsequent to phase separation the aqueous phase was extracted with 1,000 ml (7 vol) methyl-tert-butylether (MTBE) and the combined organic phases washed with 273.2 ml (2 vol) brine. Subsequently the separated organic phase was dried over sodium sulphate. Thereafter the organic phase was filtered and concentrated in vacuum at 45° C. The obtained material was dissolved twice with a minimal amount of ethyl acetate (EtOAc) and concentrated to complete dryness to afford 136 g of compound of formula (3′) with 96% yield.

### EXAMPLE 4

### Preparation of (3S,5S,8R,9S,10R,13S,14S)-3-((tert-butyldimethylsilyl)oxy)-10,13-dimethyltetradecahydrospiro[cyclopenta[a]phenanthrene-17,2′-[1,3]dioxolan]-6(1H)-one (compound of formula (4′), step d.)

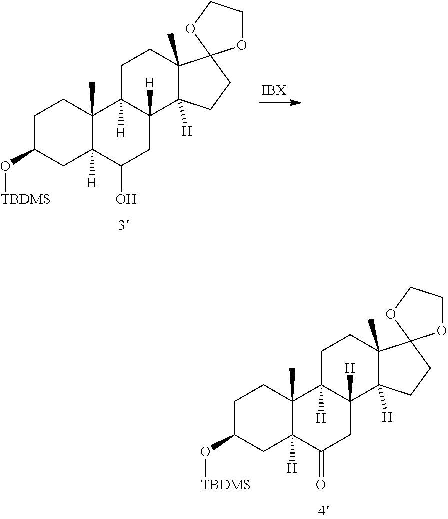

A reactor was charged with a solution of 27.9 g (1.0 eq.) compound of formula (3′) in 83.7 ml (3 vol) ethyl acetate (EtOAc), 167.4 ml (6 vol) dimethyl sulfoxide (DMSO) and 33.6 g (2.0 eq.) 2-iodobenzoic acid (IBX). The reaction mixture was mixed at ambient temperature until completion of the reaction, which was monitored by chromatography. Subsequently the reaction mixture was extracted with 558 ml (20 vol) methyl-tert-butylether (MTBE) and 279 ml (10 vol) H₂O. The contents were separated by filtration and the obtained cake washed with 50 ml (1.8 vol) methyl-tert-butylether (MTBE). The liquid phases where collected and the aqueous phase removed. Thereafter, the organic phase was washed two times each with 239.5 ml (5 vol) aqueous sodium bicarbonate (NaHCO₃ aq.) and one time with 55.8 ml (2 vol) brine. Subsequently, the organic phase was dried over sodium sulphate, separated by filtration and concentrated to dryness under vacuum at 60° C. to afford 26.1 g compound of formula (4′) with 83% yield.

### EXAMPLE 5

### Preparation of tert-butyl(((3S,5S,8R,9S,10R,13S,14S)-10,13-dimethyl-6-((trimethylsilyl)oxy)-1,2,3,4,5,8,9,10,11,12,13,14,15,16-tetradecahydrospiro-[cyclopenta[a]phenanthrene-17,2′-[1,3]dioxolan]-3-yl)oxy)dimethylsilane (compound of formula (5′), step e.1))

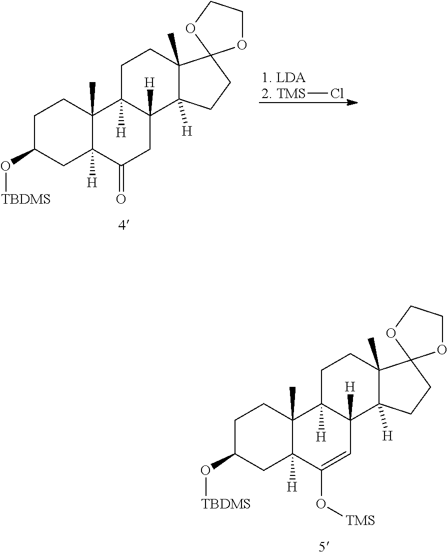

A reactor was charged with 47.6 g (1.0 eq.) of compound of formula (4′) and 238 ml (5 vol) tetrahydrofuran (THF) anhydrous. The mixture was cooled to −70° C., slowly charged with 77.14 ml (1.5 eq.) lithium diisopropylamide (LDA) and mixed for 3 hours at −70° C. While keeping the temperature in the range of −70° C. to −65° C. 15.61 ml (1.2 eq.) trimethylsilyl chloride (TMS-Cl) were slowly added to the reaction mixture. Upon completion of the dosing the reaction mixture was allowed to warm to ambient temperature during a period lasting for approximately 10 hours. Thereafter, the reaction mixture was quenched with 952 ml (20 vol) aqueous sodium bicarbonate (NaHCO₃ aq.) and extracted with 952 ml (20 vol) methyl-tert-butylether (MTBE). Subsequently, the organic phase was washed with 95.2 ml (2 vol) brine and dried over sodium sulphate. Thereafter the organic phase was filtered, rinsed with 100 ml (2.1 vol) methyl-tert-butylether (MTBE) and concentrated to dryness under vacuum at 30-35° C. to afford 55.2 g compound of formula (5′) with 100% yield.

### EXAMPLE 6

### Preparation of (3S,5S,8R,9S,10R,13S,14S)-3-((tert-butyldimethylsilyl)oxy)-7-hydroxy-10,13-dimethyltetradecahydrospiro[cyclopenta[a]phenanthrene-17,2′-1,3dioxolan]-6(1H)-one (compound of formula (6′), step e.1))

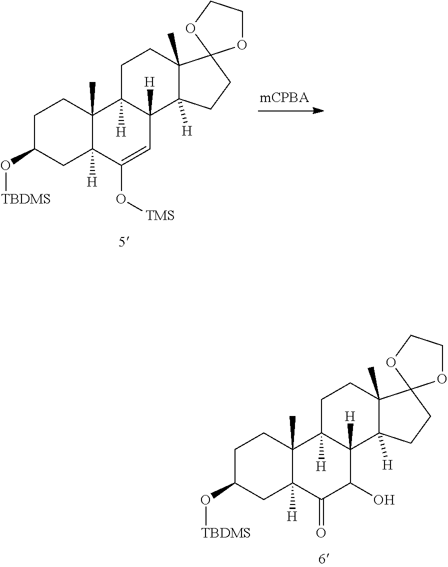

A reactor was charged with 54.0 g (1.0 eq.) of compound of formula (5′) in 270 ml (5 vol) dichloromethane (DCM) and allowed to cool to −20° C. While keeping the temperature below 0° C., 26 g (1.15 eq.) meta-Chloroperoxybenzoic acid (mCPBA) were slowly charged to the reaction mixture and further mixed at a temperature of 0° C. until completion of the reaction, which was monitored by chromatography. Thereafter 120 ml (2.2 vol) aqueous sodium bicarbonate (NaHCO₃ aq.) were charged to the mixture to adjust the pH to 7-8 at while keeping the temperature at 10° C. 1,080 ml (20 vol) methyl-tert-butylether (MTBE) were charged to the reaction mixture followed by an extraction which was performed with 270 ml (5 vol) H₂O. The phases were separated and the organic phase washed with 270 ml (5 vol) aqueous sodium bicarbonate (NaHCO₃ aq.), 270 ml (5 vol) brine. Thereafter the organic phase was dried over sodium sulphate, filtered, rinsed with 100 ml (˜2 vol) methyl-tert-butylether (MTBE) and concentrated to dryness under vacuum at 30° C. to afford 55 g of compound of formula (6′) with 100% yield.

### EXAMPLE 7

### Preparation of (1,2R,5S)-5((tert-butyldimethylsilyl)oxy)-2-((3aS,4R,5S,7aS)-4-formyl-7a-methyloctahydrospiro[indene-1,2′-[1,3]dioxolan]-5-yl)-2-methylcyclohexane-1-carboxylic acid (compound of formula (7′), step f.)

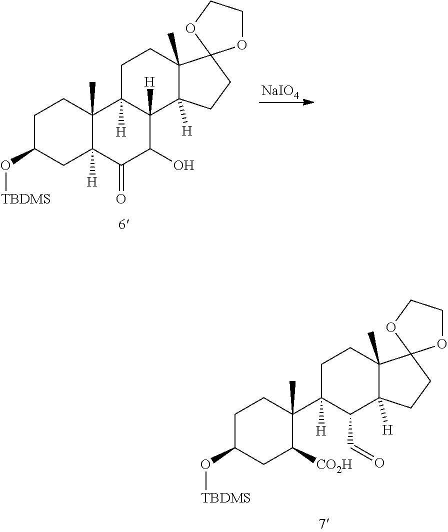

A reactor was charged with a solution of 62.7 g (1.0 eq.) compound of formula (6′) in 376 ml (6 vol) methanol followed by charging of 56 g (2 eq.) sodium periodate (NalO₄). The reaction mixture was mixed at ambient temperature until completion of the reaction which was monitored by chromatography and lasted for a period of approximately 10 hours. Thereafter the reaction mixture was charged with 3,000 ml (˜48 vol) methyl-tert-butylether (MTBE) and extracted with 4,000 ml (˜64 vol) H₂O. Subsequently the separated organic phase was washed with 125.4 ml (2 vol) brine, dried over 100 g, filtered, and concentrated to dryness under vacuum to afford 55.7 g of compound of formula (7′) with 86% yield.

### EXAMPLE 8

### Preparation of (3aS,4R,5S,7aS)-4-(aminomethyl)-5-((1R,2S,4S)-4-hydroxy-2-(hydroxymethyl)-1-methylcyclohexyl)-7a-methyloctahydro-1H-inden-1-one 4-methylbenzenesulfonate (compound of formula (10′), step g-i.)

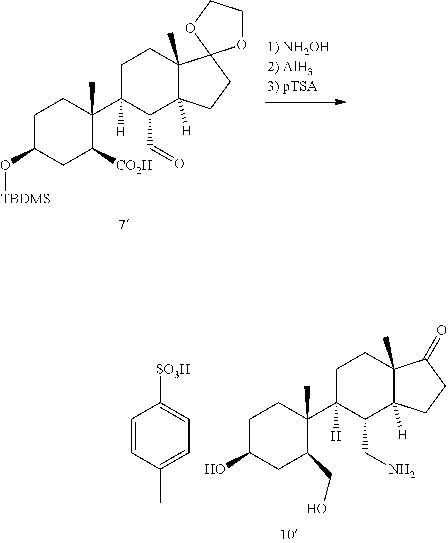

A solution of 5.0 g (1.0 eq.) compound of formula (7′) in 15.0 ml (3 vol) tetrahydrofuran (THF) and 0.61 ml (1.1eq.) 50% aqueous hydroxylamine (NH₂OH 50% aq.) were mixed over a period of approximately 10 hours and subsequently charged into 80.8 ml (8 eq.) of a freshly prepared THF solution of 1M aluminium hydride (AlH₃). The reaction mixture was heated to 60° C. and mixed for 8 hours, then cooled to ambient temperature and charged with 16.2 g (5 eq.) of sodium sulfate decahydrate (Na₂SO₄.10H₂O) followed by mixing lasting 2 hours. Thereafter the reaction mixture was filtered, the obtained cake washed three times each with 20 ml (4 vol) tetrahydrofuran (THF) and three times each with 20 ml (4 vol) methanol. The filtrates were concentrated and the solvent exchanged with ethanol until ˜20-30 ml solution remained. Subsequently 2 g (1.05 eq.) of p-toluenesulfonic acid (p-TSA) monohydrate were charged to the reactor and mixed for 2 hours at 45° C. The mixture was cooled to ambient temperature and charged into 300 ml (60 vol) methyl-tert-butylether (MTBE) at vigorous mixing. The precipitates were separated by filtration, washed three times each with 20 ml (4 vol) methyl-tert-butylether (MTBE) and dried under vacuum at ambient temperature to afford 5 g of compound of formula (10′) with 100% yield.

### Example 9

### Preparation of (1S,3S,4R)-4-((3aS,4R,5S,7aS)-4-(aminomethyl)-7a-methyl-1-methyleneoctahydro-1H-inden-5-yl)-3-(hydroxymeth)-4-methylcyclohexan-1-ol (compound of formula (11′), step j.)

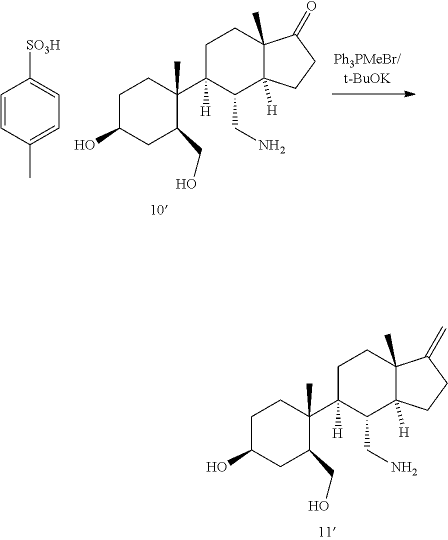

A reactor was charged with 5.0 g (1.0 eq.) compound of formula (10′) in 50.5 ml (5 eq.) potassium tert.-butoxide (KOt-Bu) (1M/THF) and the reaction mixture mixed for 20 minutes at ambient temperature. 14.4 g (4.0 eq.) of Ph₃P⁺MeBr⁻ were added and the reaction mixture mixed at a temperature of 45-50° C. until complete conversion which took approximately 2 hours, and was monitored by chromatography. Subsequently the reaction mixture was cooled to 0° C., charged with 20 ml (4 vol) H₂O, evaporated and chased twice each with 20 mil (4 vol) tetrahydrofuran (THF). The residue was suspended in 100 ml (20 vol) dichloromethane (DCM), separated by filtration and concentrated. The crude compound of formula (11′) was purified via silica gel column eluted with 9:1:0.2 DCM/EtOH/7M NH₃:MeOH to afford 1.4 g compound of formula (11′), 95% pure by HPLC. Thereafter, 40 ml of dichloromethane (DCM) were charged to compound of formula (11′) and mixed at ambient temperature for approximately 10 hours. Subsequently compound of formula (11′) was isolated by filtration, washed twice each with 10 ml dichloromethane (DCM) and dried under vacuum to afford 1.2 g of compound of formula (11′), 99.2% pure by HPLC.

### EXAMPLE 10

### Preparation of (1S,3S,4R)-4-((3aS,4R,5S,7aS)-4-(aminomethyl)-7a-methyl-1-methyleneoctahydro-1H-inden-5-yl)-3-(hydroxymethyl)-4-methylcyclohexan-1-ol acetate (compound of formula (12′), step k.)

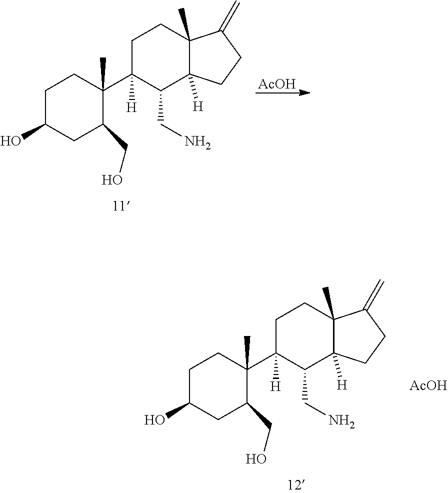

A reactor was charged with 1.0 g (1 eq.) compound of formula (11′), 10 ml (10 vol) MeOH and 0.2 g (1.1 eq.) of AcOH, concentrated in a vacuum and triturated with 20.0 ml (20 vol) CAN. The solids were separated by filtration and dried under vacuum to afford 1.18 g compound of formula (12′), 100% yield.

All examples for steps a. to i. according to the invention result in a high yield (greater or equal to 86%), step j. according to the invention results in a yield of at least 40% in combination with a high purity (>80%) for each of the steps a. to j.

1. A process for producing a compound of formula (11) 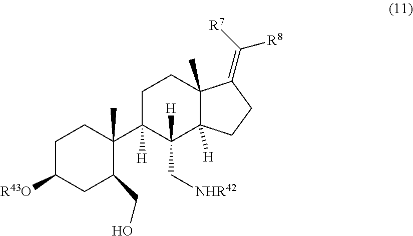
wherein R⁷ and R⁸ are each independently selected from H, halogen, alkyl, aryl, or alkylaryl, R⁴² is H or a protective group, R⁴³ is H or R³, wherein R³ is a protective group, by contacting a compound of formula (10) 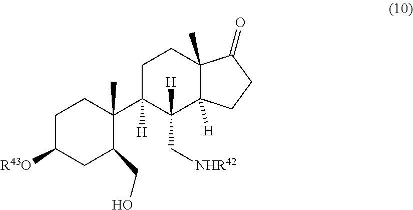
with an olefination reagent, wherein compound of formula (10) comprises a counter acid X¹ when R⁴²═H and R⁴³═H.

2. Process according to claim further comprising a step of contacting a compound of formula (3) 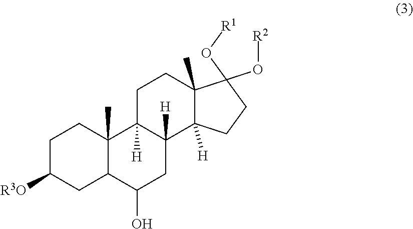
wherein R¹, R² and R³ are protective groups, with a second oxidizing agent to obtain a compound of formula (4)

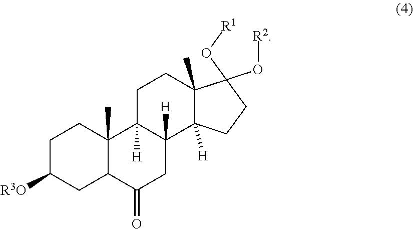

3. Process according to claim 2, further comprising the step of contacting a compound of formula (9). 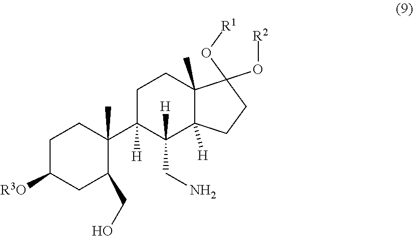
wherein R¹, R² and R³ are protective groups, with an first acid capable of forming a pharmaceutically acceptable salt to obtain the compound of formula (10).

4. Process according to claim 2, further comprising:
a) converting the compound of formula (4) to a compound of formula (7) 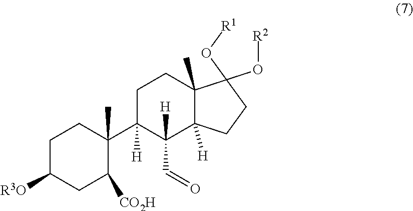
comprising the steps selected from:
iv) contacting the compound of formula (4) with a first base and a third protective reagent to form a compound of formula (5) 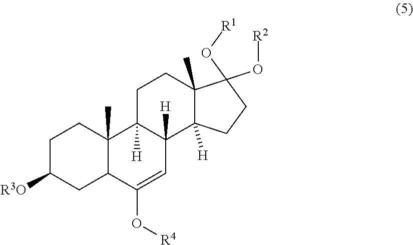
wherein R⁴ is a protective group, and
contacting the compound of formula (5) with a third oxidizing agent and optionally with a fourth protective reagent to obtain a compound of formula (6) 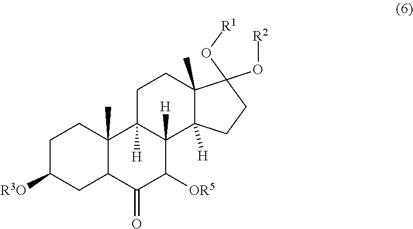
wherein R⁵ is H or a protective group; and
contacting the compound of formula (6) with a fourth oxidizing agent to obtain the compound of formula (7);
or
v) contacting the compound of formula (4) with a first base and a third oxidizing agent and optionally with a fourth protective reagent to obtain a compound of formula (6), wherein the molar ratio of the third oxidizing agent and the compound of formula (4) is at most 1.5; and
contacting a compound of formula (6) with a fourth oxidizing agent to obtain the compound of formula (7);
or
vi) contacting the compound of formula (4) with a first base and a third oxidizing agent and optionally with a fourth protective reagent to obtain the compound of formula (7), wherein the molar ratio of the third oxidizing agent and the compound of formula (4) is at least 1.5;
wherein the process further comprises the steps of:
b) contacting the compound of formula (7) with a compound of formula (7a)
NH₂—R⁶ (7a)
wherein R⁶ is either H or —OR²², wherein R²² is either H or a protective group,
to obtain a compound of formula (8) 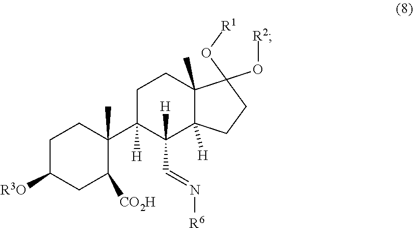
and
c) contacting the compound of formula (8) with a reducing agent to obtain the compound of formula (9).

5. A process for preparing a compound of formula (12) 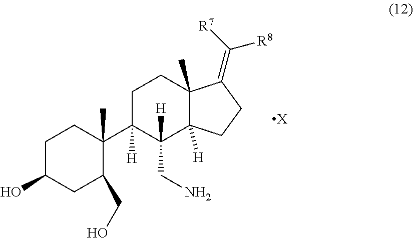
wherein R⁷ and R⁸ are each independently selected from H, halogen, alkyl, cycloalkyl, alkoxy, aryloxy, aryl, or alkylaryl and X is a counter acid, comprising the steps of claim 1, further comprising the conversion of the compound of formula (11) with a second acid capable of forming a pharmaceutically acceptable salt to obtain the compound of formula (12).

6. A process for preparing a compound of formula (4) 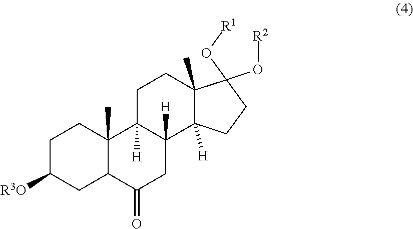
wherein R¹, R² and R³ are protective groups, comprising the step of contacting a compound of formula (3) 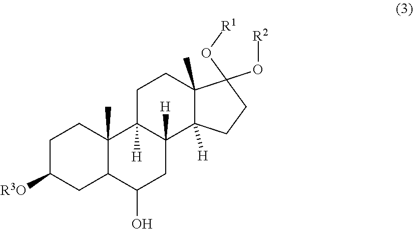
with a second oxidizing agent to obtain the compound of formula (4).

7. (canceled)

8. A compound of formula (5) 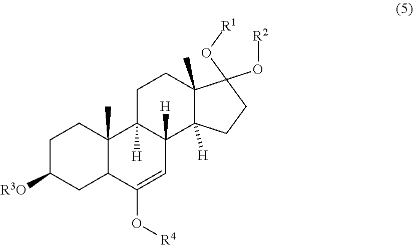
wherein R¹, R², R³ and R⁴ are protective groups.

9. A compound of formula (6) 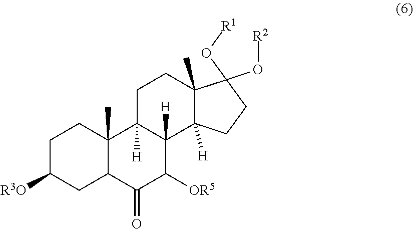
wherein R¹, R² and R³ are protective groups, and R⁵ is H or a protective group.

10. A compound of formula (7) 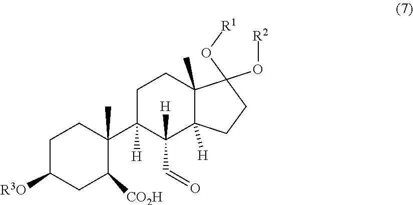
wherein R¹, R² and R³ are protective groups.

11. A compound of formula (8) 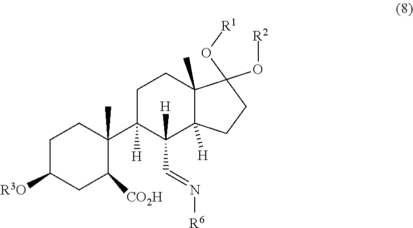
wherein R¹, R² and R³ are protective groups and R⁶ is either H or —OR²², wherein R²² is either H or a protective group.

12. A compound of formula (9) 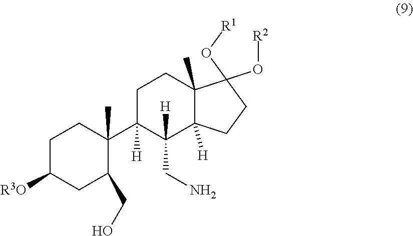
wherein R¹, R² and R³ are protective groups.

13. (canceled)

14. (canceled)

15. (canceled)
